SEQUENCE LISTING (37 sequences)

SEQ ID NO: 1 - DNA, 1530 bp, O.sativa
atggaggtggagttgccatggggcgcgcggtgcgcggcggcggcgttcttcgtgtcctcgctgtgcgtggcggcgctcggcgtcgtgctcctgctcctcaggcggtggccgtggtgcggctgccatgtctgccgcgcctacctggccgggtcgtggaggagggagttcgccaacctcggcgactggtacgccgacctgctccgccgctcgccgacgggcaccgtccacgtccacgtcctcggctgcaccgtcacggcgaacccggcgaacgtggagtacatgctcaagacgcgcttcgacaacttccccaaggggaggccgttcgccgcgctcctcggcgacctcctcggcgacggcatcttcaacgtcgacggcgacgcgtggcgccaccagcggaagatggccagcctcgagctggggagcgtcgccgtgagatcctacgcgtacaagatcgtcgcccaggaggtggaggcccgcctcatgccggtgctcgccaacgccgccgacagcggcgccgtggtcgacctgcaggacgtgttccgccgcttcgccttcgacaccatctgcaagatctccttcggcctcgacccgggctgcctcgaccgggagatgcccgtgtcggagctcgccgacgcgttcgacgccgcgtcgcggctgtccgccatgcgtggcgcggcggcgtcgccgttgctgtggaagatgaagcggtttctcaacgtcgggtcggagagggagctcaagaaggccatcaagctcatcgacgggctcgcggcggcgatgatccgggagcgccggaagcttggcgtcgcgaacagccacgacctcctgtcccggttcatggcctcctccggcgacgacgcgcgcggcgccgccgacgacaagttcctccgcgacatcgtcgtcagcttcctcctcgccggccgggacacggtgtcctccgcgctcaccactctgttcatgatcctgtccaagaaccccgacgtggcggccgccatgcgcgcggaggccggcgccgccgccggcgagagcgccgccgtcagctacgagcacctgaagcggctgaactacacccacgccgtgctgtacgagaacatgcggctgttcccgccggtgcagttcgactccaagttctgcgccgccgccgacgtgctccccgacggcacctacgtcgacggcggcgcgcgcgtcatgtaccacccctacgccatgggccgcatgccgcgcatctggggcgccgactgcgacgcgttccggccggagcggtggctcaccggcgccggcggcgcgttcgtgccggagagcctcttcaagtacccggtgttccaggccggcctccgcgtgtgcctcggcaaggagctcgccatcaccgagatgaaggcggtcagcgtcgccgtcgtgagggcattcgacgtcgaggtcgtcggcgagaatggccggtgcggcggcggcgccgccgccgcgccaagattcgtgccggggctcaccgcgtccatcagcggtgggctcccagtgaagatcagacgcgtttag

SEQ ID NO: 2 - protein, 509 aa, O.sativa
MEVELPWGARCAAAAFFVSSLCVAALGVVLLLLRRWPWCGCHVCRAYLAGSWRREFANLGDWYADLLRRSPTGTVHVHVLGCTVTANPANVEYMLKTRFDNFPKGRPFAALLGDLLGDGIFNVDGDAWRHQRKMASLELGSVAVRSYAYKIVAQEVEARLMPVLANAADSGAVVDLQDVFRRFAFDTICKISFGLDPGCLDREMPVSELADAFDAASRLSAMRGAAASPLLWKMKRFLNVGSERELKKAIKLIDGLAAAMIRERRKLGVANSHDLLSRFMASSGDDARGAADDKFLRDIVVSFLLAGRDTVSSALTTLFMILSKNPDVAAAMRAEAGAAAGESAAVSYEHLKRLNYTHAVLYENMRLFPPVQFDSKFCAAADVLPDGTYVDGGARVMYHPYAMGRMPRIWGADCDAFRPERWLTGAGGAFVPESLFKYPVFQAGLRVCLGKELAITEMKAVSVAVVRAFDVEVVGENGRCGGGAAAAPRFVPGLTASISGGLPVKIRRV

SEQ ID NO: 3 - DNA, 1560 bp, O.sativa
atgggcgcggacgccaccacgttgcacatcgcggttcacgccctagcgagctccttgcaggcgcaggttgccgccgtcttcttcgtctcggccgcgtgcacggtggccctcgcgctgctgctggctttgcagcggctgcggccgccgtggtggtgcgcgtgcccggtgtgcgaggcgtacgtgaccgcgtcgtgggcgcgcgagttcgacaacctctgcgactggtacgcgcacctgctgcgccgcgcgccggggcgaaccgtgcacgtgcacgtgctcgggaacgtgctcaccgccaacccggccaccgtcgaccacatgctgcgctgccgcttcgacaactaccccaagggtgcccccttctcggccatcctcgccgacttcctcggccgcggcatattcaacgtcgacggcgacggctggctcttccagcgcaagctcgccgcggctgagctcgcctcgccggcgatacgagccttcgcggcgaacgtcgtggcctcggaactgcggtgccgcctcatccctctgcttcgctctgcctccagctacgggagcgagaggctgctggatctccaggacgtcttccgccgcttcgcgttcgactgcatatgcaagatctccttcggcctcgaccccggctgcctcgagctttcgctgccgatatcggcgttcgcggacgcgttcgacacggcgtcgatgctctcggcccggcgagcgacggtgcccatgcatgtcgtctggaagttgaagcggctacgcaacatcggggaggagcgggagctccgcgacgcgatccgtctcgttgacgcgcttgccgccgaggtcatccggcaacgccgcaagctcggctccgccgcctcctgtgacgacctcctgtcgcgattcatgggctccatcaacgacgacaagtacctccgcgacatcgtcgtgagcttcatgctcgccggccgcgacactgtcgcctcaggtctaaccgcgttcttcttgctcctctccgaccatcccgaggtggctgccgcaatccgggacgaggtcttccgcgtcgccggaggcagggaccccacggccgcctccttcgatttcgacaagctcaaggacatgcactacgtgcacgcggcgctgtacgagagcatgcggctgttcccgccggtgcagttcgactccaagttcgccgccggcgacgacacgctcccggacggcacgttcgtggcaaagggcacacgtgtgacctaccacgcgtacgccatgggccggatggagtccctctggggccccgactgcgccgtgttcagaccggagcggtggctcagcggcggccggttcgtgccggagagcccgtacaggtacccggtcttccagggcggggtgcgcgtctgcgtaggcaaggatctcgccatcatggagatgaaggccgtcatcgtcgccgtcgtccttagcttcgacgtggaggcggtcgaccggagctcgcgccggcccaagttcgcgcctggcctcaccgccacgttcgccgggggcgtgccggtcagagtgcgccggcgagcgcactgcgcgccaagttag

SEQ ID NO: 4 - protein, 519 aa, O.sativa
MGADATTLHIAVHALASSLQAQVAAVFFVSAACTVALALLLALQRLRPPWWCACPVCEAYVTASWAREFDNLCDWYAHLLRRAPGRTVHVHVLGNVLTANPATVDHMLRCRFDNYPKGAPFSAILADFLGRGIFNVDGDGWLFQRKLAAAELASPAIRAFAANVVASELRCRLIPLLRSASSYGSERLLDLQDVFRRFAFDCICKISFGLDPGCLELSLPISAFADAFDTASMLSARRATVPMHVVWKLKRLRNIGEERELRDAIRLVDALAAEVIRQRRKLGSAASCDDLLSRFMGSINDDKYLRDIVVSFMLAGRDTVASGLTAFFLLLSDHPEVAAAIRDEVFRVAGGRDPTAASFDFDKLKDMHYVHAALYESMRLFPPVQFDSKFAAGDDTLPDGTFVAKGTRVTYHAYAMGRMESLWGPDCAVFRPERWLSGGRFVPESPYRYPVFQGGVRVCVGKDLAIMEMKAVIVAVVLSFDVEAVDRSSRRPKFAPGLTATFAGGVPVRVRRRAHCAPS

SEQ ID NO: 5 - DNA, 1563 bp, O.sativa
atggatgccaccaccaccgtgtccatggagatggaattgccgtggggcgcgcggtgcgcggggctcgccttcttcgcgttctcggtgtgcctggcggcgctcggcgtcgtgctcctggtggcgaggcggtggccgtggtgcagctgccatgtgtgccgcgcctacctgacggggtcgtgggcgagggagttcaccaacctcggcgactggtacgcgcacctgctccgccgctcgccgacgggaaccgtccacgtccacgtcctcggctgcaccgtcacggcgaacccggcgaacgtcgagcacatgctcaggacgcggttcgacaacttccccaaggggaggcccttcgccgcgctcctcggcgacctcctcggcgacggcatcttcaacgtcgacggccacgcgtggcgccaccagcggaagatggccagcctcgagctcggcagcgtcgccgtgcgctcctacgcctacaagatcatcgcccaggaggtggaggcccgcctcatgccggtgctcgccgacgccgccgaccggggcgccgtgctcgatctgcaggacgtgttccgccgcttcgccttcgacaacatctgcaagatctccttcggcctcgaccccggctgcctcgaccgggaaatgcccgtgtccgagctcgccgacgcgttcgacgccgcgtcgcgcctctccgccatgcgtggcgcagcggcgtcgccgttgctgtggagggcgaagcggttcctcaacgtcgggtcggagagggagctcaggaaggccatcaaggtcgtcgacgagctcgcggcggcgatgatccgggagcgccagaagctgggcgtcggcagcagccacgacctcctgtcgcggttcatggcctccaccggcgtcgacgacgccgccgcggacgacaagttcctccgcgacatcgtcgtcagcttcctcctcgccgggcgggacacggtgtccaccgcgctcaccacgctgttcatgctcctgtccaagaaccccgaagtcgccgccgccatgcgcgcggaggcggaggccggcgacggcggcgagacgggcgccgccatcacctacgagcacctcaagggcctccactacacccacgccgtgctgcacgagaacatgcggctgttcccgccggtgcagttcgactccaagttctgcgccgccgccgacgtgctccccgacggcacctacgtcggcggcgacgcgcgcgtcatgtaccacccctacgccatggggcggatgccgcacatctggggcgccgactacgccgcgttccggccggcgcggtggctcaccggccccggcgcgtcgttcgtcccggcgaacccgtacaaatacccggtgttccaggccggccaacgcgtgtgcctcggcaaggagctcgccgtcaccgagatgaaggcggccagcgtcgccgtcgtgagggcgttcgacgtcgaggtcgtcggcgagaatggccggagcggcggcgccgccgccgcgccacggttcgtgccggggctcaccgcgtcgatcagtggcgggctccaagtgagagtcaggcgccgcgttcacacttag

SEQ ID NO: 6 - protein, 520 aa, O.sativa
MDATTTVSMEMELPWGARCAGLAFFAFSVCLAALGVVLLVARRWPWCSCHVCRAYLTGSWAREFTNLGDWYAHLLRRSPTGTVHVHVLGCTVTANPANVEHMLRTRFDNFPKGRPFAALLGDLLGDGIFNVDGHAWRHQRKMASLELGSVAVRSYAYKIIAQEVEARLMPVLADAADRGAVLDLQDVFRRFAFDNICKISFGLDPGCLDREMPVSELADAFDAASRLSAMRGAAASPLLWRAKRFLNVGSERELRKAIKVVDELAAAMIRERQKLGVGSSHDLLSRFMASTGVDDAAADDKFLRDIVVSFLLAGRDTVSTALTTLFMLLSKNPEVAAAMRAEAEAGDGGETGAAITYEHLKGLHYTHAVLHENMRLFPPVQFDSKFCAAADVLPDGTYVGGDARVMYHPYAMGRMPHIWGADYAAFRPARWLTGPGASFVPANPYKYPVFQAGQRVCLGKELAVTEMKAASVAVVRAFDVEVVGENGRSGGAAAAPRFVPGLTASISGGLQVRVRRRVHT

SEQ ID NO: 7 - DNA, 1575 bp, O.sativa
atgggtgtcgtcgaggcagaggcgttgcatggggcggtggaggccctggctgggtcgctgcagccgcacgtggccaccgccttcttcgtgttctcggcgtgcacggtggccctcgccgcgctccttgccgtggtgcgcctgcgcccgccgtggtggtgcgactgcaccgtgtgcgaggccttcctgacggcgtcgtgggcgggagagttcgacaacctctgcgactggtacgcccacctgctgcgcacctcgccggcgcagaccgtgcacgtgcacgtcctccgcaacgtgctcaccgccaacccggtcaccgtcgaccacgtgctccgcgcccgcttcgacaactatcccaagggcgctcccttctccgccatcctcgccgacttcctcggccgcgggatatttaacgtcgacggtgacgcgtggctcttccagcgcaagctcgccgctgccgagctcgcctctccggcgctccgcgccttcgcggcgcgtgtcgtggcctccgagctgaggtgccgcctcatccctctgctccactctgcttcacgtgagggcaacggcaaggtgctcgacctgcaagacatgttccgccgcttcgccttcgacagcatatgcaagatctcgtttggcctcgaccctggctgcctcgagctgtcgatgcctgtatcgacgctcgtggaagcgttcgacacggcgtcgacactctccgcgcggcgggcgacggtgcccatgcagataatctggaggctgaagcggttcttgaacgtcggggacgaaaggaagctccgggacgcggtccgtttggtcgacgcgctcgcggcggaggtcatccgacaacggcggaagcttggcggcgctgccaccggcagcgacctcctttcacgcttcatgggctccatcgacgacgacaagtacctccgcgacatcgtcgtcagcttcatgctcgccggccgcgacaccatcgcctcggcgctcaccgccttctttctgctcctctccgatcacccagaggtcgccaccgccatccgagacgaggtcgcccgcgtcacgggcgacggcaaccgaaccatggccgccacattcgacaagctcaaggacatgcactatgtgcacgccgcgatgtacgagagcatgcgcctgttcccgccagtgcagttcgattccaagtttgccgccggcgacgatacgctgccggacggcacggtcgtcgcgaagggcacccgggtgacctaccacgcctacgccatgggccggatggagtccgtatggggccccgactgcgccgagttccggccggagcgatggctccgggacggccggttcgtcccggagagcccgtaccgttacccggtgttccaggcgggcgttcgcgtctgcatcggcaaggagctagccctcatggagatgaaagccgtcatcgtcgccgtcgtccggagcttcgacatcgaggcgatcgcgcggagctcgcggcggcccaagttcgcgccgggcctgaccgccacgttcgcaggcggcttgccggtgagagtgcgccggcggcgagcacgtgcgagcgggcacaacccgccaatttaa

SEQ ID NO: 8 - protein, 524 aa, O.sativa
MGVVEAEALHGAVEALAGSLQPHVATAFFVFSACTVALAALLAVVRLRPPWWCDCTVCEAFLTASWAGEFDNLCDWYAHLLRTSPAQTVHVHVLRNVLTANPVTVDHVLRARFDNYPKGAPFSAILADFLGRGIFNVDGDAWLFQRKLAAAELASPALRAFAARVVASELRCRLIPLLHSASREGNGKVLDLQDMFRRFAFDSICKISFGLDPGCLELSMPVSTLVEAFDTASTLSARRATVPMQIIWRLKRFLNVGDERKLRDAVRLVDALAAEVIRQRRKLGGAATGSDLLSRFMGSIDDDKYLRDIVVSFMLAGRDTIASALTAFFLLLSDHPEVATAIRDEVARVTGDGNRTMAATFDKLKDMHYVHAAMYESMRLFPPVQFDSKFAAGDDTLPDGTVVAKGTRVTYHAYAMGRMESVWGPDCAEFRPERWLRDGRFVPESPYRYPVFQAGVRVCIGKELALMEMKAVIVAVVRSFDIEAIARSSRRPKFAPGLTATFAGGLPVRVRRRRARASGHNPPI

SEQ ID NO: 9 - DNA, 1521 bp, Arabidopsis thaliana
atggcatttcttctgagttttttgatactagcgtttcttataaccatcatcttctttttatcttcatcttcaactaaaaaagtccaagaaaacaccacatatggaccaccatcgtatccactcattggctccatactctctttcaacaaaaaccgtcatcgtcttcttcaatggtacaccgagcttctccgtctctctccgtctcaaaccatcttagttcctctcctcggcaaccgtcgcaccatcatcacaacaaacccattaaacgtcgagtacattctcaaaacaaacttctttaacttccctaaaggtaaaccttttactgatcttcttggtgatcttctcggtggaggcatattcaatgtcgacggtcactcatggagctcgcagcgcaaactcgctagccacgagttctccactcgctcgcttaggagttttgcttttgaggttcttaaagacgaagtggagaaccgtctagtcccggttttatccacggcggctgatgttggtacgaccgtggatttgcaagatgttttgaaacgttttgcttttgatgttgtttgtaaagtctcgttgggttgggatccggattgtttggatctaacccgacccgttaatccacttgtggaggcttttgacaccgctgctgagattagtgctcgccgtgccacggagcctatttacgctgtttggaaaacgaagcgtgtgttgaacgtgggaagcgagaggaagcttagggaagcgatcaggaccgtgcacgtgttggtctctgagattgtcagagctaagaagaagagtcttgagatcggaaccggagcagaagcaaagcaagatcttttgtcgaggtttctagccgccggccacaacggcgaagcagtgagggacatggttattagttttatcatggcgggaagagatacaacgtcagcagcgatgacgtggctgttttggttgttgactgagaacgatgacgtggagcggaagattttagaagaagtggacccgttggtgagtttagggttagggtttgaggatttgaaagagatggcttacacgaaggcttgtctatgtgaagccatgaggctatatcctccagtttcgtgggactctaaacatgctgcaaacgacgacgttttgcctgatgggacacgtgtcaagagaggagacaaagtgacctatttcccatacggtatggggaggatggagacgctatggggtacggattcagaagagtttaacccgaaccggtggtttgattctgaaccggggagtacacgaccggttttgaaacctattagtccctacaaatttccggtttttcaagccgggccgagggtttgtgtagggaaagagatggcatttatgcagatgaaatacgtcgttggttctgttttgagtcggtttgagattgtgccggttaacaaagaccggccggtttttgttcctcttttaacggctcatatggctggtgggcttaaggtgaagatcaagagaagaagtcacatccttaacaacgtttaa

SEQ ID NO: 10 - protein, 506 aa, Arabidopsis thaliana
MAFLLSFLILAFLITIIFFLSSSSTKKVQENTTYGPPSYPLIGSILSFNKNRHRLLQWYTELLRLSPSQTILVPLLGNRRTIITTNPLNVEYILKTNFFNFPKGKPFTDLLGDLLGGGIFNVDGHSWSSQRKLASHEFSTRSLRSFAFEVLKDEVENRLVPVLSTAADVGTTVDLQDVLKRFAFDVVCKVSLGWDPDCLDLTRPVNPLVEAFDTAAEISARRATEPIYAVWKTKRVLNVGSERKLREAIRTVHVLVSEIVRAKKKSLEIGTGAEAKQDLLSRFLAAGHNGEAVRDMVISFIMAGRDTTSAAMTWLFWLLTENDDVERKILEEVDPLVSLGLGFEDLKEMAYTKACLCEAMRLYPPVSWDSKHAANDDVLPDGTRVKRGDKVTYFPYGMGRMETLWGTDSEEFNPNRWFDSEPGSTRPVLKPISPYKFPVFQAGPRVCVGKEMAFMQMKYVVGSVLSRFEIVPVNKDRPVFVPLLTAHMAGGLKVKIKRRSHILNNV

SEQ ID NO: 11 - DNA, 1488 bp, Arabidopsis thaliana
atgttactaatcatatcattcaccatcgtctccttcttcttcatcataatattctcactctttcacttactcttccttcaaaaactacgttactgcaactgcgagatctgtcatgcttatctcacttcaagctggaaaaaagacttcatcaatctcagtgattggtacactcatcttctccggcggtctccgacgtcaacgatcaaagtacacgtcttaaacagcgttatcacagcgaatccatcaaacgttgaacatatcctcaaaaccaatttccacaattacccaaaaggcaaacaattctctgttatcctcggtgatcttttaggccgtggaatcttcaattccgatggtgacacgtggcgtttccagagaaaactcgcgagtttggaacttggaagtgtttctgtaagagttttcgctcatgagattgttaagaccgagatcgagacacgtcttcttccgatattgacttcgttttccgacaatcctggttctgttttggatctacaagatgtgtttagaagattctcgtttgatacgattagtaaattgtcgtttgggtttgacccagattgtctccggttaccttttccgatatcggaattcgccgtcgctttcgatacggcgtcgttgttatcggcgaaacgagctttagctccgtttcctctgttatggaaaaccaaaaggcttttaagaattggttctgagaaaaagcttcaagaatctatcaatgtgataaaccggttagctggtgatttaatcaagcaacggaggttaaccggtttgatgggtaaaaatgatttgatttctagattcatggcggttgtggcggaggacgatgatgagtatctccgagacatcgttgtgagttttctattggctggtcgggatacggtagctgctggtttaaccggatttttctggttattgacccgtcatccggaagtggagaaccggatccgggaagaactggaccgggttatggggaccggttttgactcagtcactgctcgttgcgatgaaatgagagagatggattatttgcatgcgtcgctgtacgagagcatgagattgtttccgccggttcaattcgattccaaattcgctttaaacgatgacgttttgtctgatggtacatttgtgaatagtgggactagagtcacgtaccatgcttatgcaatgggtcggatggaccggatctggggcccggattacgaagagtttaaaccagagaggtggttggataacgaaggtaagttccggcccgagaatccggttaagtacccggttttccaggccggagctagggtttgtatcgggaaagagatggctatcatggagatgaaatccatagctgtggcaatcattaggaggttcgaaacacgggtcgcaagtcccgaaacaaccgagacactccggtttgcgcctggtttaactgcgacggttaacggtggattgccggttatgatccaagaaaggagttag

SEQ ID NO: 12 - protein, 495 aa, Arabidopsis thaliana
MLLIISFTIVSFFFIIIFSLFHLLFLQKLRYCNCEICHAYLTSSWKKDFINLSDWYTHLLRRSPTSTIKVHVLNSVITANPSNVEHILKTNFHNYPKGKQFSVILGDLLGRGIFNSDGDTWRFQRKLASLELGSVSVRVFAHEIVKTEIETRLLPILTSFSDNPGSVLDLQDVFRRFSFDTISKLSFGFDPDCLRLPFPISEFAVAFDTASLLSAKRALAPFPLLWKTKRLLRIGSEKKLQESINVINRLAGDLIKQRRLTGLMGKNDLISRFMAVVAEDDDEYLRDIVVSFLLAGRDTVAAGLTGFFWLLTRHPEVENRIREELDRVMGTGFDSVTARCDEMREMDYLHASLYESMRLFPPVQFDSKFALNDDVLSDGTFVNSGTRVTYHAYAMGRMDRIWGPDYEEFKPERWLDNEGKFRPENPVKYPVFQAGARVCIGKEMAIMEMKSIAVAIIRRFETRVASPETTETLRFAPGLTATVNGGLPVMIQERS

SEQ ID NO: 13 - DNA, 1795 bp, Glycine max
cccgttatatatatcacacattccaacccttcttctccacagttccatccttcactccaaaaatttactactaaagagatgagttttgcaggttttctttctcttcaatcaatgtctgctacctgcggcttcttcttcttctccttcacccttctcttctccttcttctccttcctcgtcttcatctccaggatgaagccctggtgtaactgcgatctttgcaagagctacttgacggtgagttggctgaaaaagttccccaatctctgcgactggtacactcatctcttgcgtaaatccccaaccggcaccatccacgttcacgtgctggggaacaccatcacctcgaatccccacaacgtggagcacattctcaagacgaatttccaaaactaccccaagggaaaacccttctccaccatcctcggcgatcttttgggaagaggaatcttcaacgtcgacggcgaatcgtggaagttccagcgcaagatggcgagtctcgaactcggcagcgtggcgattcggacgtacgcgatggaattagtcaacgaggagatccacgcgcgactgatcccgatcatggaatcaaccgcgcgtggcgaattaaactccgtctgcgttttggatttgcaagatatactacgaagattctcgttcgacaacatatgcaaattctcgttcgggctcgacccgggttgtctcctaccgaatctcccagtctcggacctcgccgtcgcgtttgacctggcctcaaagctctccgccgaacgcgccatgaacgcgtcgccgtttatttggaaactaaaacgactcctcaacataggatccgagaagaaactgagagaaacaattaacgtcgtaaacgacgtcgcaaaagagatgataaagcagagaagagaaatggggttcaagacgcggaacgaccttctctcgcggttcatgggatcaatagacgacgacgtttatttaagagacattgtggtgagttttctattagcgggtcgtgacaccatcgctgccgggttaacgggcttttttatgttgttatcgaaaagcccggaggtggaggagttgattagggaggaggtgggccgggtgatgggcccgggccaggagtttccttcgtttgagcaaatccgcgaaatgcattacctgaacgcggcgattcacgacagcatgaggctgttcccaccgattcagttcgattcgaagttcgccacggaggatgacgtgttgccagatggcactttcgtgagaaagggcagtagggtaacttaccacccttacgcgatgggccgaatggaaaatatttgggggcccgattgtcttgacttcaggcccgaaaggtggctgcgtgatggggtgttcgtgccagagtgtccctttaagtaccctgtttttcaggctggtgtaagggtgtgtttggggaaggacttggctttgatggagatgaagtccgttgtggttgcgttggttcgcaggtttgatattcgggtcgcccaaccggatcaggagccccggttcgcgcccggtctaaccgctactttgagaggggggtttccggttcgggtttgtgaaaggaaatgaatagataaaggagttcacaacatgaatgtgaatggaatcatgaaatgaatgagtgaatgaaggaacagttttcgtgtaaataatataatgttgcagttaaatcgaacgtttttctttttttttctcctcttttaaacaggcgtctttttgaatttgcgtttatgtttattattgcccagtgaagtaaatactaaagccattgcaaatt

SEQ ID NO: 14 - protein, 502 aa, Glycine max
MSFAGFLSLQSMSATCGFFFFSFTLLFSFFSFLVFISRMKPWCNCDLCKSYLTVSWLKKFPNLCDWYTHLLRKSPTGTIHVHVLGNTITSNPHNVEHILKTNFQNYPKGKPFSTILGDLLGRGIFNVDGESWKFQRKMASLELGSVAIRTYAMELVNEEIHARLIPIMESTARGELNSVCVLDLQDILRRFSFDNICKFSFGLDPGCLLPNLPVSDLAVAFDLASKLSAERAMNASPFIWKLKRLLNIGSEKKLRETINVVNDVAKEMIKQRREMGFKTRNDLLSRFMGSIDDDVYLRDIVVSFLLAGRDTIAAGLTGFFMLLSKSPEVEELIREEVGRVMGPGQEFPSFEQIREMHYLNAAIHDSMRLFPPIQFDSKFATEDDVLPDGTFVRKGSRVTYHPYAMGRMENIWGPDCLDFRPERWLRDGVFVPECPFKYPVFQAGVRVCLGKDLALMEMKSVVVALVRRFDIRVAQPDQEPRFAPGLTATLRGGFPVRVCERK

SEQ ID NO: 15 - DNA, 1890 bp, Glycine max
ctttaatcaatttttaatagtagcatggcactacagtgaaccaaacccattatatgtatcacacattccaaccctccttctccacatttcctttcttttactccccccccccccccccataaaaaaatatagagagagatgagttttcaaggttttctttctctccaatcaatgtctgcaacctgcggcttcttgttcttcgccttcacccttctgttttccttcttctcgttcctcgtttttgtctcgaggatgaagccgtggtgtaactgcgatgtttgcaagagctacttgacggtgagttggctgaacaagtttccaaatctctgcgactggtacactcatcttctgcgcaaatccccaaccggcaccatccacgtgcacgtgctggagaacaccataacctcgaaccccgtcaacgtggagtacattctcaagacgaatttccaaaactaccccaaggggaaaccgttctccgtcatcctcggcgatcttcttggaagaggaatcttcaacgtcgacggcgaatcgtggaagttacaacgcaagatggcgagtttggagctcgggagcgtcgctattcgaacgaacgcgatggaattggtcaacgaggagatccacgcgcgtctgatcccattcataatgggatcagtcacgcacgacgaattaaacgactccgtctgcgttttggatttgcaagatatattacgaagattctcgttcgacaacatatgcaaattctcgttcggtctcgacccgggctgtctcctaccgaatctcccagtctcaaacctcgccgacgcgtttgacttggcctcgaagctttccgcggaacgcgccatgaacgcgtcgccgtttatttggaaactaaaacgactcctcaacgtaggttccgaaagaaaactgagagaagcaatcaacgttgtaaacgacgtcgctaatgagatgatcaaacagagaatagaaatggggttcaacacgcggaacgaccttctctcgcggttcacgggatccataaacgacgacgtttatttaagagacattgtggtgagttttctcttagcgggccgtgacaccatcgcctccgggttaacgggcttttttatgttgttgtctaaaagcccggaggtggaggagttgattagagaggaggcgggccgggtggtgggcccgggccaggagtttccttcgtttgaacaaatccgcgaaatgcattacctgaacgcggcgattcacgagagcatgaggctgttcccaccgattcagttcgattcgaagttcgccacggaggatgacgtgttgccagatggcacttttgtgagaaagggtagtagggtaacttaccacccttacgcgatgggccgaatggagaacatttgggggcccgattgtctcgagttcaggcccgaaagatggctgcgtgatggagtgttcgtgccagcgtgtccttttaagtaccctgtttttcaggctggggtaagggtgtgtttggggaaggacttggctttgatggagatgaagtccgttgtgcttgcgttggttcggaggtttgatattcgggtcgttcaatcgggccaggagccccggttcgagcccggtttaaccgctactttgagaggcgggttgccggttcgggttcgggtttgtgaaaggaaatgaatagcaagtatgtggttgataaagtcataaagccataaaggagttcacaactagaatgaaatcatgaaatcaatgaggggatggaagaaccgttttcgtgtaaataataatgttgcagttgaattgaaagttttctttttccccctcttttaagctggtttcttgctgaatttgcgtttatatttattattacccggtgaagtaaatactaaatactataagcgttgcaaa

SEQ ID NO: 16 - protein, 506 aa, Glycine max
MSFQGFLSLQSMSATCGFLFFAFTLLFSFFSFLVFVSRMKPWCNCDVCKSYLTVSWLNKFPNLCDWYTHLLRKSPTGTIHVHVLENTITSNPVNVEYILKTNFQNYPKGKPFSVILGDLLGRGIFNVDGESWKLQRKMASLELGSVAIRTNAMELVNEEIHARLIPFIMGSVTHDELNDSVCVLDLQDILRRFSFDNICKFSFGLDPGCLLPNLPVSNLADAFDLASKLSAERAMNASPFIWKLKRLLNVGSERKLREAINVVNDVANEMIKQRIEMGFNTRNDLLSRFTGSINDDVYLRDIVVSFLLAGRDTIASGLTGFFMLLSKSPEVEELIREEAGRVVGPGQEFPSFEQIREMHYLNAAIHESMRLFPPIQFDSKFATEDDVLPDGTFVRKGSRVTYHPYAMGRMENIWGPDCLEFRPERWLRDGVFVPACPFKYPVFQAGVRVCLGKDLALMEMKSVVLALVRRFDIRVVQSGQEPRFEPGLTATLRGGLPVRVRVCERK

SEQ ID NO: 17 - DNA, 1845 bp, Glycine max
tcacacgtacgcacacttatatacatatatatcctcagaaccctgccctgtattcaatctttatccataatccatcaatggagctagagcttgaatcttttacactgtgcactctgatgcagctcttcaccaacatcccctttggcagcacactgtgtctttcactggccattatcatcatcaccctcgtgatcctcactaccttttcttcatctctcttccttcgctggccaaaaataatcactttcttctgtagctgcgaaacatgccaggcctatctcacttcaggttggtccaaaaccttcgacaacctttgcgattggtacgctcacctcctccgaaactcgccgacaaaaaccatccacatccacgtcctccgaaacaccatcaccgccaaccccgacaacgtggaatacatgctcaagaccaggttccacaacttccccaagggaaaaacgttctccaccatcctcggcgatttcctcggccgaggaatcttcaacgtcgacggagaatcctggagcttccagaagaagatggcgagtcttgaactcagcaagaactcgataagatccttcgccttcgaggtcgtcaaattcgaaatcaaagaccgactcatccctctccttgttctgtccaaacaaaatgactgtgttttggatttacaagacgttttcaagagattctcgttcgacagcatttgtagattctccttcgggttggaccctatgtgtctggagctgtctctgcctatatcggagtttgctatgtcctttgacttggcgtcgaaactgtccgcggagagagccatgtcggtgtcccctctaatatggaagatcaaacggttcctcaacgtgggctccgagaagaagctgagaaaagctattaaaatgattgacatcttagccagagaagtcataagacaaagaagaaaaatggggttttcatcaatttcacctcataaggacgacttgttatcacggttcatgagaaccatcactgatgacacatacttaaaagacattatcgtaagtttcttgctagccggtcgtgataccgtggcttcggctctaacaagcttcttttggctcttagcaaaacacccagaagtggaatcccaaattcttctagaagcagagcaagtaattggatcagactataacaaagaccttacaagctatgaagagcttcaacaactgcactatttacaagctgcggcgaatgaaagcatgagactttatcctccgattcaattcgattccaagttttgcttagaagatgatgtgttgcctgatgggacttttgtgaagagaggaactagggttacttaccatccctacgcgatggggcgtttggaggagatatggggttcggattgctttgagtttaggccagagaggtggttgaaggaaggacttttttgccctcagaatccgttcaagtatccggtttttcaagcggggataagggtgtgtttggggaaagaaatggcactgttggagctcaagagtgtggcgctttcgttgcttcggaggttccacatcgaattggcggcaccaccaccgcatcatcatcatcacagtaccccacgtttctcgccaggcctcactgcaacttttagttgcggtcttcgcgtaaaggtacgtgaacaaggaacccaacaacgacaacacactactcaacctcttcatcattcatagtgattatgaacgaatattcataattatcccacctggctgttgacaagggagaaaataatataaaagaaattatgaatctttgagaaaattggatgatatacacttcggttcttaattattacttaattcttgcataaaagcacta

SEQ ID NO: 18 - protein, 540 aa, Glycine max
MELELESFTLCTLMQLFTNIPFGSTLCLSLAIIIITLVILTTFSSSLFLRWPKIITFFCSCETCQAYLTSGWSKTFDNLCDWYAHLLRNSPTKTIHIHVLRNTITANPDNVEYMLKTRFHNFPKGKTFSTILGDFLGRGIFNVDGESWSFQKKMASLELSKNSIRSFAFEVVKFEIKDRLIPLLVLSKQNDCVLDLQDVFKRFSFDSICRFSFGLDPMCLELSLPISEFAMSFDLASKLSAERAMSVSPLIWKIKRFLNVGSEKKLRKAIKMIDILAREVIRQRRKMGFSSISPHKDDLLSRFMRTITDDTYLKDIIVSFLLAGRDTVASALTSFFWLLAKHPEVESQILLEAEQVIGSDYNKDLTSYEELQQLHYLQAAANESMRLYPPIQFDSKFCLEDDVLPDGTFVKRGTRVTYHPYAMGRLEEIWGSDCFEFRPERWLKEGLFCPQNPFKYPVFQAGIRVCLGKEMALLELKSVALSLLRRFHIELAAPPPHHHHHSTPRFSPGLTATFSCGLRVKVREQGTQQRQHTTQPLHHS

SEQ ID NO: 19 - DNA, 1726 bp, Zea mays
cattttcctctccttgtctctggcgccatggaagcagctgatgccttgccctgggcagcgcagtgtgcgggcatgaccgtcctcgcctcctccctctgcttggtggcgctggccctggtgctcctgctccttcggcgctggccgtggtgcagctgccatgtctgccgggcgtacctgacggggtcgtgggagcgggacttcaccaacctcggtgactggtacgcgcacctcatccgggagtcggccaccggcaccgtccacgtccacgtgctgggctgcaccgtcaccgccaacccggccaacgtggagtacatgctcaagaccaacttcgacaacttccccaagggcgagaccttcgcaacgctccttggggacctcctcggctgcggcatcttcaacgtggacggccacgcgtggcgctaccagcgcaagatggccagcctggagctcggcagcgtggccgtgcggtcctacgccttcgggatcatcgccagggaggtggaggcccggctcatgccgctcctcgccggcgccgccgacgccggcgcggtggtcgacctgcaggacgtgttccgacgcttcgccttcgacaccatctgcaagatctccttcggcctcgacccgggctgcctcgagcgggagatgcccatgtccaagctcgccgacgccttcgacaccgccacgcgcctctgcgccatgcgcggcgccgccgcgtcgccgttgctctggagggccaagcgcctgctcaacgtcggctcggagagggagctcagggaggccatccgtctggtcgacgagctcgcggctgccgtgatccgggagcgccgcaagctgggcgtcgccagcagccacgacctcctgtcgcgcttcatggtggcctcggccggcggcgacgcgcaccccggcgttgacgataagtacctccgcgacatcgtagtcagcttcctcctcgcggggcgcgacaccgtgtcctcggcgctcaccacgctcttcatgctcctgtccaagaacccggccgtggtggccgccatgcgcgccgaggccggggacggcgacggcgacggcgactcagcgccgctcacctacgagcacctcaagcgcctgcactacacccacgcggtgctgtacgagaacatgcgcctgttcccgccggtgcagttcgactccaagttctgcgccgggcccgacgtgctccccgacggcatctacgtgtccggcggcacgcgcgtgatgtaccacccctacgccatgggccggatgccgcgcatctggggcgccgaccacgacgcgttccgcccggaccgctggctcacgggcgccggcggttccttcgtccccgagagcctgtacaagtacccggtgttccaggcgggcctccgcgtgtgcctcggcaaggagctcgccatcacggagatgaaggcggtcggggtggccgtggtgagggcgttcgacgtccacgtcgtcggggacagtggcagcgccgcctgcgcgccaaagttcgtgtcggggctcaccgcgtccattagcggcgggctcccagtgaggattagtagaagagttcgaagctagagcgaggttcttgttttcgttcgtaaattaaaaaacaagtagagactactagatcgatagagctgtttcgcggataatgtacatagactaattaaaacaaattgcaagttgtaattccatatatatatttcgtatatatgggattttcttatgctcc

SEQ ID NO: 20 - protein, 513 aa, Zea mays
MEAADALPWAAQCAGMTVLASSLCLVALALVLLLLRRWPWCSCHVCRAYLTGSWERDFTNLGDWYAHLIRESATGTVHVHVLGCTVTANPANVEYMLKTNFDNFPKGETFATLLGDLLGCGIFNVDGHAWRYQRKMASLELGSVAVRSYAFGIIAREVEARLMPLLAGAADAGAVVDLQDVFRRFAFDTICKISFGLDPGCLEREMPMSKLADAFDTATRLCAMRGAAASPLLWRAKRLLNVGSERELREAIRLVDELAAAVIRERRKLGVASSHDLLSRFMVASAGGDAHPGVDDKYLRDIVVSFLLAGRDTVSSALTTLFMLLSKNPAVVAAMRAEAGDGDGDGDSAPLTYEHLKRLHYTHAVLYENMRLFPPVQFDSKFCAGPDVLPDGIYVSGGTRVMYHPYAMGRMPRIWGADHDAFRPDRWLTGAGGSFVPESLYKYPVFQAGLRVCLGKELAITEMKAVGVAVVRAFDVHVVGDSGSAACAPKFVSGLTASISGGLPVRISRRVRS

SEQ ID NO: 21 - DNA, 1682 bp, Zea mays
agcaccgcacaacacaaacgtccccacacatttcccctaccatctccctcttcttccacgagctaattctagacagaacacaagcgcccgccatcgccatggaagcaacagcagactcgttgccgtgggcggcgcagtgcgcgggcgcggcactgttcgccgccgccctctgcgtgacggctctgggcgtcgtgctggtgttcctccggcggtggccctggtgcagctgccacgtgtgccgggcgtacctgacggggtcgtgggcgcgggacttcaccaacctcggcgactggtacgcgcacctgctccgggagtcgcccacgggcaccatccacgtgcacgtgctcggctgcaccgtcaccgccaacccggccaacgtcgagtacatgctgaggaccaacttcgacaacttccccaaggggaaggccttcgcggcgctgctcggggacctcctcggcggcggcatcttcaacgtggacggccacgcctggcgccaccagcgcaagatggccagcctcgagctcggcagcgtcgccgtccggtcctacgcgttcgggatcgtggcgcaggaggtggagtcccgcctcgtgccggtgctcgccgccgccgccgacgctggcgcggtcgtggacctgcaggacgtgttccggcgcttcgcgttcgacaccatctgcaagatctccttcggcctggaccccggatgcctggagcgggagatgcccgtgtcgaggctcgccgacgcgttcgacgtcgccacgcggctgtgcgccatgcgcggcgccgcggcgtcgccgctagtgtggagggccaagcgcctgctgaacgtcgggtccgagagggagctccgggaggccatcgcgctcgtcgacgagctcgcggccgccgtgatccgggagcgccggaagctgggagtcgccggcagccacgacctcctgtcccgcttcatggcctcggccggcgccggcgacggcgacgacaagtacctccgcgacatcgtcgtgagcttcctgctggccgggcgcgacaccgtgtcctccgcgctgaccacgctgttcatgctcctgtccaagaacccgggcgtggcggccgccatgcgcgcggaggccggcgacggcgagtccgccgcgccgctcacctacgagcacctcaagggcctgcactacacccacgcggtgctgtacgagaacatgcggctgttcccgccggtgcagttcgactccaagttctgcgcggggcccgacgtgctccccgacggcacctacgtggccggcggcacgcgcgtgacgtaccacccctacgcaatgggccggatgccgcgcatctggggcgccgaccacggcgccttccgccccgaccggtggctcaccggagccggcgcctcgttcgtcccagagagcctgtataggtacccggtgttccaggcgggcctccgcgtctgcctcggcaaggagctcgccgtcaccgagatgaaggtggtcgctgtggccgtggtgagggcgttcgacgtcgaggtggtcggggacagtggcgccaccgcctgcgcgcccaagttcgtgtcggggctcaccgcgtccatcagcggcgggcttccggtgagggtcaggaggagagttgttcgatagagattcttgggctggcggtggcgggtggtgtgtgtgtgtttttccaaaaaaaaaaaaaaa

SEQ ID NO: 22 - protein, 507 aa, Zea mays
MEATADSLPWAAQCAGAALFAAALCVTALGVVLVFLRRWPWCSCHVCRAYLTGSWARDFTNLGDWYAHLLRESPTGTIHVHVLGCTVTANPANVEYMLRTNFDNFPKGKAFAALLGDLLGGGIFNVDGHAWRHQRKMASLELGSVAVRSYAFGIVAQEVESRLVPVLAAAADAGAVVDLQDVFRRFAFDTICKISFGLDPGCLEREMPVSRLADAFDVATRLCAMRGAAASPLVWRAKRLLNVGSERELREAIALVDELAAAVIRERRKLGVAGSHDLLSRFMASAGAGDGDDKYLRDIVVSFLLAGRDTVSSALTTLFMLLSKNPGVAAAMRAEAGDGESAAPLTYEHLKGLHYTHAVLYENMRLFPPVQFDSKFCAGPDVLPDGTYVAGGTRVTYHPYAMGRMPRIWGADHGAFRPDRWLTGAGASFVPESLYRYPVFQAGLRVCLGKELAVTEMKVVAVAVVRAFDVEVVGDSGATACAPKFVSGLTASISGGLPVRVRRRVVR

SEQ ID NO: 23 - DNA, 1747 bp, Zea mays
aaaagccgaatccgaagccaagagccgtatcgacatggaactctccctgacctcatctgctgctctgcagttcgtcctgctcctccccctgttctctgtgctctgcatactctacctgcgccaggatcccaagaagcagcctcgcgctcatggcctcaaggtctacccgctcgtcggtaccctcccgcacttcgtcaagaaccagcgccgcttcctccactggtccaccgacgtcctcaagcgcgacccctcgcacaccctgtcgttcaaggcgctcggcctcaccggcggcgtcatcacggctaacccggccaacgtcgagcacatcctcaagaccaacttcgccaactaccccaagggcgagctgacggtgtctatgatcgaggacttcctcgggcacggcatcttcaactccgacggggagcagtggctgtggcagcgcaaggccgccagctacgagttcagcaagcgctcgctcaggagcttcgtggtcgacgccgtccggttcgaggtcgtggagcggctgctgccgctgctcgaccgggcgcagcgcgacgggcggacgctggacgtgcaggaagtgctggagcgcttcgcgctcgacaacatctgccgcgtggcgttcggcgacgacccggcgtgccttgccgaggaggagggcatggcgcagaggcagagcgccgagttcatgcgcgccttcaacgacgcgcagagcgccatcatggcccggttcatgtcgcccgtgaagtcgctgtggcgcctcaagaggctgctgaacgtggagcccgagcggcggatgcgcgaggcggtccacaccgtccacggctacgccgacaggatcatccgcgagcgccgggcgaggggcgaggcggccgagcccgcgcgcgacgacgacttcctgtcgcgcttcgccgcgagcggcgagcacagcgacgagagcctccgggacgtggtcaccaacttcctcctcgccgggcgggacacgacgtcgtcggcgctgacgtggttcttctggctggtctccacgcggcccgacgtggaggagaagatcgtgcgcgaggtccgcgcggtgcggcgtctgggaagcgcgggcgcggcggcgccggcgttcagcttcgacgagctgcgggacatgcagtacctccacgcggccatcaccgagtccatgcggctgtacccgccggtggccatggacacgcacagctgcaaggaggacgacttcctccccgacggcacgttcgtcggcaaagggtggctgacgacctactccgcgttcgcgatgggccgcgtggaggacatatggggcgcggactgcgaggagttcaggccggagcggtgggtcagcgaggacggcgcgttccgcccggagagcccgttcaagtacccagtcttccacgccgggccacggatgtgcctcggcaaggagatggcgtacatccagatgaagtccattgctgcatgcgtgctcgagaggttcagcttccagttcgtcggcggcggggagcggccggggctcgtgctctcgctcacgctgcggatgcagggcggtctgcccatgaaagtggtcaccaggacgaactcggctctaggctagactttccacaccagacagtatccatctcaatacacatacgattggacggtttagtgagattggggtgtaatttcacttagttttcgagctgatccatgtcaacccatatctatatatgaattaaaatgaatacgagcaaaaaaaaaaaaaaaa

SEQ ID NO: 24 - protein, 519 aa, Zea mays
MELSLTSSAALQFVLLLPLFSVLCILYLRQDPKKQPRAHGLKVYPLVGTLPHFVKNQRRFLHWSTDVLKRDPSHTLSFKALGLTGGVITANPANVEHILKTNFANYPKGELTVSMIEDFLGHGIFNSDGEQWLWQRKAASYEFSKRSLRSFVVDAVRFEVVERLLPLLDRAQRDGRTLDVQEVLERFALDNICRVAFGDDPACLAEEEGMAQRQSAEFMRAFNDAQSAIMARFMSPVKSLWRLKRLLNVEPERRMREAVHTVHGYADRIIRERRARGEAAEPARDDDFLSRFAASGEHSDESLRDVVTNFLLAGRDTTSSALTWFFWLVSTRPDVEEKIVREVRAVRRLGSAGAAAPAFSFDELRDMQYLHAAITESMRLYPPVAMDTHSCKEDDFLPDGTFVGKGWLTTYSAFAMGRVEDIWGADCEEFRPERWVSEDGAFRPESPFKYPVFHAGPRMCLGKEMAYIQMKSIAACVLERFSFQFVGGGERPGLVLSLTLRMQGGLPMKVVTRTNSALG

SEQ ID NO: 25 - DNA, 1590 bp, O.sativa
ctgcttaccgccttctactccgtcttcttcctcctctctctctctctctctccctcgttgccgtcgcgactcgcgaggaagactggtgttttgggggtacagggtttggttcatcgaggcgatggagagcggagccggatccttggaggcgcggccgggcgtcctcgtcgtcggcgcgcctggcgtcggcaagagaaccatcctctcccgactgcttgcagctgaaataccggacacacatgacttgtcatcaggtgtgctgtgtcaggggtggaacattgacacaaagtactactcagcagatctttctgtttggacggcacatcttgggcaagggttttctcttgattccctccctcatctcgaccagttggatgctcttgttatggtcttcgatatgagtgatgaatcatccttccttactctccagaactgggtttctagtgttgatgttcagaggtttgaaattctgttgtgcattggaaataaagcagatcttgtgcttggccatggtgcacacgtagaatataggaggcgtatgcaaaagattggcgagtcaagtactgatccacatcctgaatatttggattttgggataaacgaaagtgaaggttgtggattgttatcagaggaagaaccacaaatagagatcagagactccgcttcacattggtgcattgaacacaatattgagtatgtcaaagcttgtgcttccaatgctgactttgataaatgtctatcggtgaatggagatagtcaaggcttagagcgactccttggagccctttctgcacacatgtggcctggaatgattctgaaatctggaaacagaataaccgttccatccttggttgagaaagaagaatctacagatgatgaatccaattacgaatttgagtacgaagttctgtcccatggatctgatgaacaatgggaattcattggtgagtcaagtacgtcaagagacttggagggattgaatgaagatacagctactcatgataaaactaatcaggttgtggatgctgatgctaattcagtggcatccaatcctcttccaaatggcagaatgacagaaactgctgaggaaaaactggtacctcagagccacaaaagcgaggttagtaatgatatggataacacacaagcagatgcttcagagaaactgcaagctggtatcccagagggtaacaagctatttgaagatgagcattacggtctagatgatctggagcggctaatgtcagagattggcaacatgcgctccaatttaaggctcatgcctgacttccaaaggaaagagatggctgccaagcttgccatgaaaatggcaaccatgtttggtgataacgacgaagagggctctgaagatatctgattcaattcacggagcccaccttttgcatgtcttcaaacatatgttcccagtttatggcgtgctgatttgtacaggcagatgggccgcaggccgtgtaggcatttccatcggctatcataattcccagtgaaattttgtaccttagtagttccatatttaccattcttactgctgtaagttgtatactgtattatttgaaattaaagttctct

SEQ ID NO: 26 - protein, 418 aa, O.sativa
MESGAGSLEARPGVLVVGAPGVGKRTILSRLLAAEIPDTHDLSSGVLCQGWNIDTKYYSADLSVWTAHLGQGFSLDSLPHLDQLDALVMVFDMSDESSFLTLQNWVSSVDVQRFEILLCIGNKADLVLGHGAHVEYRRRMQKIGESSTDPHPEYLDFGINESEGCGLLSEEEPQIEIRDSASHWCIEHNIEYVKACASNADFDKCLSVNGDSQGLERLLGALSAHMWPGMILKSGNRITVPSLVEKEESTDDESNYEFEYEVLSHGSDEQWEFIGESSTSRDLEGLNEDTATHDKTNQVVDADANSVASNPLPNGRMTETAEEKLVPQSHKSEVSNDMDNTQADASEKLQAGIPEGNKLFEDEHYGLDDLERLMSEIGNMRSNLRLMPDFQRKEMAAKLAMKMATMFGDNDEEGSEDI

SEQ ID NO: 27 - protein, 43 aa, O.sativa
DSQGLERLLGALSAHMWPGMILKSGNRITVPSLVEKEESTDDE

SEQ ID NO: 28 - DNA, 1497 bp, Arabidopsis thaliana
aaatcagaattagcatctgtttagattggatcttgatcactggcgggcgaagttggagaagattgatggaaagccgaccgggagttttggtcgtcggatcctctggcgtcggcaagcgcaccctcctctctcgattgctctctgtggaatttgaggattcatcggagtcatcatctcaaacagaagttcatggctggacgattaacactaagtattacactgcggatgtttctgtatgtatatcacatatttgcgatgaatactctctacccaatctccctaactcacatccactggtggcgttagttatggtcttcgacttgagtgagctgtcaacccttgttgctcttcaagactgggtttctcatactgacatcaacagttttgatatattgttgtgcattggtaacaaagtagatcgtgtccctcatcaccttgctcatgatgaatatagacggcgccttttgaaggcatcagacccctctagagatctgtattcggacattgatgactttgggatctccgaaactgaaggaagcagcttgttgggaagtgaagatgcatcactagatatcaggggagcatgtcttgagtggtgctctgagaataacattgagttcattgaggcttgtgcctctaatcctgattttgacaagtgtttatcagtcgatggggattctcaaggggttgaacgtctatttggtgctctttcggctcatatgtggcctgggatgattctgaaatctggtgataggataaatgaacctgtgttacctcaaggggaagagttgtcagaagaagaatctgaatatgaattggagtatgaagtgctatcagcaggctcaggtgatccatgggataatattgaagaaagatgggtttctgcaagtgaaacgcattcacatgccgatgctggcggatccacttcacaggaaaacctccatgtagaaaatagtatggtgaaaccgaataaagttgatgaagagctgctgccgtcagggtcaaggttagagctgcaaaatgacactgacagagcaatagtaacagatgaaaaacctttgggcacagaaaatgaaaaatgttacgagtttgaagatgtggaacagttgatgtcagaaataggaaacatccgggacaacttgcgattgatgcctgatttccagaggagggaaatagctgcaaatttagcgatgaaaatggcttccatgtttggaggtgacactgatgacgaggaagaacctgagtgagtctgtttatatcagatattgtaaaagatcagcgtgagctacggtttaatcctttctttatacaatctttatcaaagtccgtgtcttgttttttgatagattctggcagtaatagtcagttgcacgatgtaatacttatctgggccgacctgttatgtatcgctttactttttctttagatacgtaagagctctaaaatacagccttatacagaaaacctagttggtaacacttggcgaagcaattgttg

SEQ ID NO: 29 - protein, 393 aa, Arabidopsis thaliana
MESRPGVLVVGSSGVGKRTLLSRLLSVEFEDSSESSSQTEVHGWTINTKYYTADVSVCISHICDEYSLPNLPNSHPLVALVMVFDLSELSTLVALQDWVSHTDINSFDILLCIGNKVDRVPHHLAHDEYRRRLLKASDPSRDLYSDIDDFGISETEGSSLLGSEDASLDIRGACLEWCSENNIEFIEACASNPDFDKCLSVDGDSQGVERLFGALSAHMWPGMILKSGDRINEPVLPQGEELSEEESEYELEYEVLSAGSGDPWDNIEERWVSASETHSHADAGGSTSQENLHVENSMVKPNKVDEELLPSGSRLELQNDTDRAIVTDEKPLGTENEKCYEFEDVEQLMSEIGNIRDNLRLMPDFQRREIAANLAMKMASMFGGDTDDEEEPE

SEQ ID NO: 30 - DNA, 1722 bp, Vitis vinifera
gatattcgagggtgggatagcaggtttgtgcaaacaaacagaatctgggaagttgggcgtgaaggtggttgattcagaaacaacgaaccctaacaacaagaatggatgcgaaatctgtggagaagacgcccggaatcttgatcgtcggatcctcctccgtcggtaaacgtactctcctctcccggttgctttctgtggattttgaagatggtgctgattcctcttctgaaattcttgttcatgggtggactatcaacacaaagtactatacagctgatgtttcagtatggatggcccatcttcatgaagggttttcgatagggaacctgccaatgcttgaccagttggctgctgtggtcatggttttcgacatgaatgatctgtcatcttttgttgcacttcaagattgggtttctcacactgatattcaaaattttgacatattattatgtgttgggaacaaagtggatcttgttccaggccattcagctcatgtggagtacagaagacgactgcagcggcttggtgacttgtctgctgaaccttatttagagttctccgagtatggaatatcagaaactgaaggaagtagtttgttaggggatgaagaatcaccatgggagataagcaggtcatgtttggagtggtgcagcgaacgcaacattgaatacattgaagcttgtgcatccaacgccgattttgacaaatgtttgtcagttaatggtgattcacaaggagttgatagaatcctgggtgcactttctgctcatatgtggcctggaatgaccctgaaatctggtgataagataatggaaccatcattgcctgaaaaccaagagttgtcagaagaagaatctgattatgaaattgagtatgaaatcctctctgctggttcagctgagccatgggatgacacagacgaggtatgggtgtctgcaagtggccctactgcatccacaagtacaagcagatcactcactcagaatgttgaaatcgaagaacatgatcaagagaagaaaaacagaggcgatgcaagagagcttcaagtctcaacatcaacagctccatcacaggaagagattaatggggagatggtacccaaagtgaatgaaactgatgaagctgtggaggtagatgagggtaaacaccttaaatatgaagatcttgaacagttgatgtctgagattgggaacatgcgtgacagcttgaggttgatgcctgattttcaaaggagggaaatggctgcaaatcttgccatgaaaatggctaccatgtttggggacggcagtgacgaagagggatttgatggatgaaaccagatgttactatgaagattaacttactgctccaaatgggaattgctcatggccgttcattaaattatccagagggaggtgatgaacaatttcatttaccttattgaaacacatggtgattgaaaattttgtgttacttttgccttttgcttctctgttttactgtagaaagttgttttgagtttgaatccatgaggctgtgccttgatttaggaaaatagctggaaattcaggtatatgtcttaaatacattttgctctagagaatgggaaattagctagctggctcttacatttgcattgctggaaaacacaaaatccccaaagtcaccatcttctaatcatatgttattgcattgtgattaaaattcaatgtatccaacacatactggaaattcaggtcaaaa

SEQ ID NO: 31 - protein, 403 aa, Vitis vinifera
MDAKSVEKTPGILIVGSSSVGKRTLLSRLLSVDFEDGADSSSEILVHGWTINTKYYTADVSVWMAHLHEGFSIGNLPMLDQLAAVVMVFDMNDLSSFVALQDWVSHTDIQNFDILLCVGNKVDLVPGHSAHVEYRRRLQRLGDLSAEPYLEFSEYGISETEGSSLLGDEESPWEISRSCLEWCSERNIEYIEACASNADFDKCLSVNGDSQGVDRILGALSAHMWPGMTLKSGDKIMEPSLPENQELSEEESDYEIEYEILSAGSAEPWDDTDEVWVSASGPTASTSTSRSLTQNVEIEEHDQEKKNRGDARELQVSTSTAPSQEEINGEMVPKVNETDEAVEVDEGKHLKYEDLEQLMSEIGNMRDSLRLMPDFQRREMAANLAMKMATMFGDGSDEEGFDG

SEQ ID NO: 32 - DNA, 1735 bp, Populus trichocarpa
taaatcaggaattctgcagcagaacttgtgttttaactgatttttataggttttcaagaaattttcaaagatcaacgaaaaatgagtgaagaaactaaatcatcagattttacatcacttgagacccgaccaggcatcttgtttatcggatcctctaacgttggcaaacgcactctcctctcaaggttgctgtcaattgatttggaagacgcatttgactcatcaaaccaagttttctcacatggatggagtattgatacaaagtattacacagctgatgtttctctatggatagctcatcttcatgaaggattttcaattggatcacttccaatttacaacaagcttgctgctttggttatggtttttgacctgagtgatttgtcatcttttgttgcacttaaggattgggtagctggaaatgatattagcaagtttgagatattgttgtgtattggaaacaaagttgatcggattccaggtcatcctgttcatgctgaatatagaagacagctaaggaaaattggagagtcaggatcttttgatgatctgaatatagagcttgatgagtttggaatttctgagattgaaggaagtagtttgttgggtgatgaagaagagccatcacgggagatcaagaggtcatgtattgaatggtgcactgatcatggaattgaatacattgaagcttgtgcttccaatgttgattttgacaaatgtttattggttgatggggactcgcaaggagtagaacgtctatatggtgctctttctgctcatatgtggccaggaatgatcctgaaatctgacaataagataatccaaccaacattacctgataaagaagaaatctcagaagatgaatctgattatgaattagagtatgaagtgctatctggtggctcagctgaaccatgggatgacacatatggaggatgggtttctgcaaatggtagaagtgccattccagatgaagggggatcagttactgaaaataattatgtcaaagaatgtgagaatgaaaatagagaaaagtttgataaagagatgcagccctcatcatcggcaacagaattgcaaggtgataagagagcggtgcctgatgtacaagaaccttgtgaaaatggagagtcggatgaagttacgccttttgactttgaggatttggagcagttgatgtctgagatagggaatgtgcgtgataacttaaggttgatgcctgattttcaaaggagggaaatggctgcgaatctggccatgaaaatggctgccatgtttggaggtggcagtgatgttgatgatgaggaagttgagtgatcagatgtatttgcatgaactaagtggtttaatttgtatccttgtgaagtctagttcgtggtgattttgggatggtaatgcgtccaaccatctacaaggtaaaactacaagataaaacaaatgtactgaaattttgtgttggccactcgattccattggtggatttactctccagcttcataaggtggttgccgagatgacccatattttgtgctcaatcatgtttagatattgggcgttagatgatctccccatgtttgtagaagatacgagtccaaggagtcttgtcttgttattataggaacggaacccttaattatgtggattgatattccaacacttgcctaatggttttgttgtaacaagtagctaaccagatgaatttggtttctatattcaatttaggacttgcaacaaa

SEQ ID NO: 33 - protein, 411 aa, Populus trichocarpa
MSEETKSSDFTSLETRPGILFIGSSNVGKRTLLSRLLSIDLEDAFDSSNQVFSHGWSIDTKYYTADVSLWIAHLHEGFSIGSLPIYNKLAALVMVFDLSDLSSFVALKDWVAGNDISKFEILLCIGNKVDRIPGHPVHAEYRRQLRKIGESGSFDDLNIELDEFGISEIEGSSLLGDEEEPSREIKRSCIEWCTDHGIEYIEACASNVDFDKCLLVDGDSQGVERLYGALSAHMWPGMILKSDNKIIQPTLPDKEEISEDESDYELEYEVLSGGSAEPWDDTYGGWVSANGRSAIPDEGGSVTENNYVKECENENREKFDKEMQPSSSATELQGDKRAVPDVQEPCENGESDEVTPFDFEDLEQLMSEIGNVRDNLRLMPDFQRREMAANLAMKMAAMFGGGSDVDDEEVE

SEQ ID NO: 34 - DNA, 1679 bp, Hordeum vulgare subsp. Vulgare
ggtcttctctctgactctctcctggctccctcgcccggcaaggaagagaagcgctagggtacagggttaggggggcgaggccgccatggaggaggggggagacggatccctcgaggcgcggcccggcgtcctgctcgtcggcgcccccggcgtgggcaagcgaaccatcctctcccggttgcttggggcggaagtgccggacacatccgacttgtcctccggtgtgctgtgtcaggggtggaaaattgacaccaagtactactctgctgatatttccatttggacagcacatcttgaggaagggttttctctcggctcgctccctcatctggaccagttggctgctctcattatggtcttcgatatgaatgatgaatcatccttagttaccctccgaaactgggtcggtaatattgatgttcagaggtttgaagttctgctctgcataggaaataaagcagatcttgtgcctggccatggtgcgcatgtggaatacaggaggcgcatgcaaaagattggcgagtcatgtagtgatccacatcctgaatatttggattttgggataaatgaaaatgaaggttgtggtttgttatcagaggaagagccgcaaatagagatcagagattccactttgaagtggtgcattgaccataatattgagtatattgaagcttgtgcttccaacgctgattttgacaaatgcctatcagtggacggtgatagccaaggcctagagcgactatttggagccctttctgcacacatgtggcccggaatgattctgaaatctggaaacaaaataactgctccatccttggtcgaaaaagatgaatctacagatgatgaggattatgaatttgactatgatgtcctatcccatgcatctgatgagcaatgggaattcgtcggtgaatcgagtacatcaagaagcttggaaggattagatgaagctaaatctttgcaggacaacacacagcaagttgagaacggcaatgctagttcttcggcgtacaaccctcttccagatgacagatctacagagcctgctgaagaaattcctgtaactcagagtcaaacacctgaagatagtagtaatcatgtggataacactgaagcaaatacttctgaggaccagcgaactgacaccccagaggttgatacgctgcttgaggatgatcactatggcgtagatgatctggagagactaatgtcagagattggcaacatgcgctccaacctgaggctcgttcctgacttccagaggagggagatggctgccaagatagccatgaaaatggcaaccatgtttggtgatagtgacgatgaaggcttccatgctgtctaagccaaattgcttatcttcacacaggattacaggttatggcaagcctatttgtattgggagatcggcatcatgccacctagggcatttttataaaatcctgatgccatttcccactcacacaaattttgtacctcatagttcttccagatatttagccatttaccttctgtagagttattcatactttctacctttataggttttgtgctgtatcattaagactttgatttctcttaggttcttgaatcaacgagctattgtatatgtaacttatcaaatattgtgttactgttgagttactcaaaatatgagttataaattattgtatcaaatt

SEQ ID NO: 35 - protein, 419 aa, Hordeum vulgare subsp. Vulgare
MEEGGDGSLEARPGVLLVGAPGVGKRTILSRLLGAEVPDTSDLSSGVLCQGWKIDTKYYSADISIWTAHLEEGFSLGSLPHLDQLAALIMVFDMNDESSLVTLRNWVGNIDVQRFEVLLCIGNKADLVPGHGAHVEYRRRMQKIGESCSDPHPEYLDFGINENEGCGLLSEEEPQIEIRDSTLKWCIDHNIEYIEACASNADFDKCLSVDGDSQGLERLFGALSAHMWPGMILKSGNKITAPSLVEKDESTDDEDYEFDYDVLSHASDEQWEFVGESSTSRSLEGLDEAKSLQDNTQQVENGNASSSAYNPLPDDRSTEPAEEIPVTQSQTPEDSSNHVDNTEANTSEDQRTDTPEVDTLLEDDHYGVDDLERLMSEIGNMRSNLRLVPDFQRREMAAKIAMKMATMFGDSDDEGFHAV

SEQ ID NO: 36 - DNA, 1829 bp, Glycine max
atgacttgcgtgcggctagcgtaggagaaccctagcgaagaagacgagaacgagatcctctatacctctctctcccgtaagtggaggttcagccgttggatcatacaaaatggctctgataatctaaatgaattaaagtttcggtgtctcgcacgcacgcacgcttcatggatgacatgaacgagttgaggaagaagagaaaaaccgcagacgccgagtgagagccgaaacttctctcatggaatcgcaaacggatctgctgaaccgacccggaatcttcttaattggatcctctaacgtcggcaaacgcactctcctatctcgattgacctctgttgatgttgaaggtgctgcttttgattcggcttctcaagtgaatgtccatgggtggactattaacaataagtactacactgctgatgtttctatctggatggctcatctcaatgacgatttttctgctgctaacgtgcccgtatttcgacgaatgactgcgctggtgatggtttttgacatgaatgaaccatcctccctagcagcacttcgagaatgggtatcccacactgatattcagaactttgagatattgctgtgtattggaaacaaagtggacctggttactggtcacccagcccatgctgaatacagaagacggttgctgaaacttgaagactctgctgcagatctttattcagaagagtatggaatatctgaattggagggaaccagtttattgggggatgaagaaccgtcatgggacatcagaagatcctgcttggaatggtgcactgatcacaatattgagttcattgaggcatgtggttcaaatgctgattttgacaaatgcttgtccatagatggtgatttacaaggagttgagcgtctctacggtgcactttctgctcatatgtggcctggaatggtattgaaatctggcgataggataaatcaaccatcatttcctgagaaagaagagttatcttcagaagaatctgattatgaacaagagtatgaagtcttatcagctggctcagccgattgggatgaaagtgaacaaggatgggtttctgcatcgtccttaggtgcagggggatcagctcctcaaaacaatcctggcacagatggtcaacatgaggatgaaattaaatctgataaagagtctcatcccacaacctcaagccctacaattcaggatgagagtgacaaggatgtggtgcacaatatcatggattccaaaggcgatgggaaggaaaacgaggctaaatgtctcgaattagaggatttggaacagttaatgtctgagattgggaatatgcgtgcaggcttaagattgatgcccgattttcaaagaagggatatggctgcaaagctagccatgaaaatggcttccatatttgggggagagagtgatgaagaggaaatttagtagatgttggatgttagctcaatgattctgttatagctgtaaggatagatagatcatagatgaatgccattgttggtaattttgttatgagccatcacatggaaggagacgaacaatctctgtttgcacaaggatcaggggttccttcatgtttctgccgtttatgctactctggtcactgctgatcatgatttaagtttaattactcctgtttttgtcatacactgtttaatagtccgtatcttatggagcagaacattttgtttttcttcaaaatttcatggtaatttacatggtgttttgaaaaactgtggcaggcagtagttgttacttgctgcaatgttatttgaaaaattaaacaataagtcaataacaatgaatatatgttatattagacttttaa

SEQ ID NO: 37 - protein, 395 aa, Glycine max
MESQTDLLNRPGIFLIGSSNVGKRTLLSRLTSVDVEGAAFDSASQVNVHGWTINNKYYTADVSIWMAHLNDDFSAANVPVFRRMTALVMVFDMNEPSSLAALREWVSHTDIQNFEILLCIGNKVDLVTGHPAHAEYRRRLLKLEDSAADLYSEEYGISELEGTSLLGDEEPSWDIRRSCLEWCTDHNIEFIEACGSNADFDKCLSIDGDLQGVERLYGALSAHMWPGMVLKSGDRINQPSFPEKEELSSEESDYEQEYEVLSAGSADWDESEQGWVSASSLGAGGSAPQNNPGTDGQHEDEIKSDKESHPTTSSPTIQDESDKDVVHNIMDSKGDGKENEAKCLELEDLEQLMSEIGNMRAGLRLMPDFQRRDMAAKLAMKMASIFGGESDEEEI

(19) United States
(12) Patent Application Publication
(10) Pub. No.: US 2017/0298378 A1
(43) Pub. Date: Oct. 19, 2017

(54) GENES TO PROVIDE ENVIRONMENTAL STRESS TOLERANCE AND THE USE THEREOF

(71) Applicants: National University Corporation Nagoya University, Nagoya-shi (JP); National Agriculture and Food Research Organization, Tsukuba-shi (JP)

(72) Inventors: Shin Takeda, Nagoya-shi (JP); Tsukaho Hattori, Nagoya-shi (JP); Ken-Ichi Kurotani, Nagoya-shi (JP); Hiroaki Ichikawa, Tsukuba-shi (JP)

(73) Assignees: National University Corporation Nagoya University, Nagoya-shi (JP); National Agriculture and Food Research Organization, Tsukuba-shi (JP)

(21) Appl. No.: 15/399,214

(22) Filed: Jan. 5, 2017

Related U.S. Application Data
(60) Provisional application No. 62/275,697, filed on Jan. 6, 2016.

(51) Int. Cl. C12N 15/82; C12N 9/02

(52) U.S. Cl. CPC C12N 15/8273; C12N 9/0071; C12N 15/8271

(57) ABSTRACT

It is intended to provide a gene capable of conferring more practical environmental stress tolerances to a plant and the use thereof. The present invention provides a plant having enhancement in one or two or more genes selected from the group consisting of a second gene selected from a gene group consisting of a first gene subgroup consisting of Os12g0150200 gene, Os01g0858350 gene, Os05g0445100 gene, Os11g0151400 gene, AT3G48520 gene, and AT2G27690 gene, and a gene functionally equivalent to any of these genes and a second gene subgroup consisting of Os04g0584800 gene and a gene functionally equivalent to this gene.

### TECHNICAL FIELD

The present specification relates to a gene capable of conferring environmental stress tolerances and the use thereof.

### BACKGROUND ART

Plants are used as crops in agriculture while their propagation and growth are protected and managed. The yields and quality of crops are largely influenced by climate change. In recent years, factors responsible for climate change have been expanded globally. Thus, it has been desired to secure stable food supply, irrespective of climate change.

Examples of various stresses that may cause climate change include high temperatures, low temperatures, dryness, flood, and salinity.

As plants tolerant to such stresses, for example, a transformed plant allowed to exhibit salt tolerance is disclosed (Patent Literature 1). Furthermore, a transformed plant allowed to exhibit dry stress tolerance is also disclosed (Patent Literature 2).

Meanwhile, one of the plant hormones induced when plants received some stress is jasmonic acid.

### CITATION LIST

### Patent Literature

### [Patent Literature 1] JP Patent Publication (Kokai) No. 2004-329210 A (2004)

### [Patent Literature 2] JP Patent Publication (Kohyo) No. 2014-520527 A (2014)

### SUMMARY OF INVENTION

### Technical Problem

Although the prediction of climate change is possible to some extent, it is very difficult to perfectly predict the climate change. In the actual cultivation of crops, the crops are often exposed to a plurality of environmental stresses at the same time. Accordingly, the crops need to possess tolerances to not only one stress but a plurality of stresses. However, it is considered difficult to prepare a plant that possesses such tolerances to a plurality of stresses. Only very few cases have been realized.

According to the present inventors, conventional results of evaluating stress tolerances at a laboratory level routinely used do not always correlate with actual stress tolerances. In short, even if positive results are obtained in the evaluation at a laboratory level, opposite results may be obtained in further evaluation at a practical level.

Although jasmonic acid is induced under stress, the roles of the jasmonic acid and its conjugate under stress have not yet been well-known.

The present specification provides a gene capable of conferring more practical environmental stress tolerances to a plant and the use thereof.

### Solution to Problem

The present inventors used rice full-length cDNA-overexpressing (FOX) individual rice lines to newly identify a plurality of genes introduced in a plurality of FOX rice lines that exhibit salt tolerance and the like. As a result of various stress tolerance tests using FOX rice lines for these candidate salt tolerance genes, the present inventors were further able to identify genes capable of contributing to salt stress tolerance at a more practical level or also exhibiting tolerances to other environmental stresses. The present specification provides the following means based on these findings.

(1) A plant having the increased expression of one or two or more genes selected from the group consisting of a second gene selected from a gene group consisting of a first gene subgroup consisting of Os12g0150200 gene, Os01g0858350 gene, Os05g0445100 gene, Os11g0151400 gene, AT3G48520 gene, and AT2G27690 gene, and a gene functionally equivalent to any of these genes and a second gene subgroup consisting of Os04g0584800 gene and a gene functionally equivalent to this gene.
(2) The plant according to (1), wherein the plant has an enhanced environmental stress tolerance.
(3) The plant according to (2), wherein the plant has enhanced salt stress tolerance.
(4) The plant according to (3), wherein the plant further has an enhanced additional environmental stress tolerance.
(5) The plant according to any of (1) to (4), wherein the plant has the increased expression of the first gene.
(6) The plant according to (5), wherein the first gene is selected from the group consisting of at least Os12g0150200 represented by SEQ ID NO: 1 and a gene functionally equivalent to this gene.
(7) The plant according to any of (1) to (6), wherein the plant has the increased expression of the second gene.
(8) The plant according to (7), wherein the plant has the increased expression of at least Os04g0584800 gene represented by SEQ ID NO: 25.
(9) A plant having a nucleic acid encoding a protein introduced therein or having the increased expression of the nucleic acid, wherein

the protein is any of the following (a) to (f):

(a) a protein comprising an amino acid sequence represented by any of SEQ ID NOs: 2, 4, 6, 8, 10, and 12;

(b) a protein comprising an amino acid sequence derived from the amino acid sequence represented by any of SEQ ID NOs: 2, 4, 6, 8, 10, and 12 by the deletion, substitution, addition, or insertion of one or more amino acids, and having (i) the activity of converting active jasmonic acid to inactive jasmonic acid or (ii) the activity of enhancing the environmental stress tolerances of plants;

(c) a protein comprising an amino acid sequence having 90% or higher identity to the amino acid sequence represented by any of SEQ ID NOs: 2, 4, 6, 8, 10, and 12, and having (i) the activity of converting active jasmonic acid to inactive jasmonic acid or (ii) the activity of enhancing the environmental stress tolerances of plants;

(d) a protein encoded by a polynucleotide consisting of a nucleotide sequence represented by any of SEQ ID NOs: 1, 3, 5, 7, 9, and 1;

(e) a protein encoded by a polynucleotide having 90% or higher identity to the nucleotide sequence represented by any of SEQ ID NOs: 1, 3, 5, 7, 9, and 11, and having (i) the activity of converting active jasmonic acid to inactive jasmonic acid or (ii) the activity of enhancing the environmental stress tolerances of plants; and

(f) a protein encoded by a polynucleotide hybridizing under stringent conditions to a polynucleotide consisting of the nucleotide sequence represented by any of SEQ II) NOs: 1, 3, 5, 7, 9, and 11 or a nucleotide sequence complementary thereto, and having (i) the activity of converting active jasmonic acid to inactive jasmonic acid or (ii) the activity of enhancing the environmental stress tolerances of plants.

(10) A plant having a nucleic acid encoding a protein introduced therein or having the increased expression of the nucleic acid, wherein

the protein is a protein described in any of the following (a) to (f):

(a) a protein comprising the amino acid sequence represented by SEQ ID NO: 26;

(b) a protein comprising an amino acid sequence derived from the amino acid sequence represented by SEQ ID NO: 26 by the deletion, substitution, addition, or insertion of one or more amino acids;

(c) a protein comprising an amino acid sequence having 90% or higher identity to the amino acid sequence represented by SEQ ID NO: 26;

(d) a protein encoded by a polynucleotide consisting of the nucleotide sequence represented by SEQ ID NO: 25;

(e) a protein encoded by a polynucleotide consisting of a nucleotide sequence having 90% or higher identity to the nucleotide sequence represented by SEQ ID NO: 25; and

(f) a protein encoded by a polynucleotide hybridizing under stringent conditions to a polynucleotide consisting of the nucleotide sequence represented by SEQ ID NO: 25 or a nucleotide sequence complementary thereto.

(11) The plant according to (10), wherein the plant has an enhanced environmental stress tolerance.
(12) The plant according to any of (1) to (11), wherein the plant is a dicotyledon.
(13) The plant according to (12), wherein the plant is soybean.
(14) The plant according to any of (1) to (11), wherein the plant is a monocotyledon.
(15) The plant according to (14), wherein the plant is a plant of the family Poaceae.
(16) The plant according to (15), wherein the plant is rice.
(17) The plant according to (15), wherein the plant is sugarcane.
(18) The plant according to (15), wherein the plant is corn.
(19) An expression vector for conferring environmental stress tolerances to a plant,

the vector comprising one or two or more genes selected from a gene group consisting of a first gene subgroup consisting of Os12g0150200 gene, Os01g0858350 gene, Os05g0445100 gene, Os11g0151400 gene, AT3G48520 gene, and AT2G27690 gene, and a gene functionally equivalent to any of these genes and a second gene subgroup consisting of Os04g0584800 gene and a gene functionally equivalent to this gene.

(20) A vector comprising any gene selected from the group consisting of the following (a) to (f):

(a) a gene encoding a protein comprising an amino acid sequence represented by any of SEQ ID NOs: 2, 4, 6, 8, 10, 12, and 26;

(b) a gene encoding a protein comprising an amino acid sequence derived from the amino acid sequence represented by any of SEQ ID NOs: 2, 4, 6, 8, 10, 12, and 26 by the deletion, substitution, addition, or insertion of one or more amino acids, and having the activity of enhancing the environmental stress tolerances of plants;

(c) a gene encoding a protein comprising an amino acid sequence having 90% or higher identity to the amino acid sequence represented by any of SEQ ID NOs: 2, 4, 6, 8, 10, 12, and 26, and having the activity of enhancing the environmental stress tolerances of plants;

(d) a gene consisting of a nucleotide sequence represented by any of SEQ ID NOs: 1, 3, 5, 7, 9, 11, and 25;

(e) a gene consisting of a polynucleotide having 90% or higher identity to the nucleotide sequence represented by any of SEQ ID NOs: 1, 3, 5, 7, 9, 11, and 25, and encoding a protein having the activity of enhancing the environmental stress tolerances of plants; and

(f) a gene consisting of a polynucleotide hybridizing under stringent conditions to a polynucleotide consisting of the nucleotide sequence represented by any of SEQ ID NOs: 1, 3, 5, 7, 9, 11, and 25 or a nucleotide sequence complementary thereto, and encoding a protein having the activity of enhancing the environmental stress tolerances of plants.

(21) A transformant comprising an expression vector according to (19) or (20).
(22) A transformed plant comprising an expression vector according to (19) or (20).
(23) A method for conferring environmental stress tolerances to a plant, comprising the step of:

increasing the expression of one or two or more genes selected from a gene group consisting of a first gene subgroup consisting of Os12g0150200 gene, Os01g0858350 gene, Os05g0445100 gene, Os11g0151400 gene, AT3G48520 gene, and AT2G27690 gene, and a gene functionally equivalent to any of these genes and a second gene subgroup consisting of Os04g0584800 gene and a gene functionally equivalent to this gene, the one or two or more genes being endogenous or foreign genes.

(24) A method for preparing a plant having an enhanced environmental stress tolerance, comprising the step of increasing the expression of any gene selected from the group consisting of the following (a) to (f) in a plant:

(a) a gene encoding a protein comprising an amino acid sequence represented by any of SEQ ID NOs: 2, 4, 6, 8, 10, and 12;

(b) a gene encoding a protein comprising an amino acid sequence derived from the amino acid sequence represented by any of SEQ ID NOs: 2, 4, 6, 8, 10, and 12 by the deletion, substitution, addition, or insertion of one or more amino acids, and having (i) the activity of converting active jasmonic acid to inactive jasmonic acid or (ii) the activity of enhancing the environmental stress tolerances of plants;

(c) a gene encoding a protein comprising an amino acid sequence having 90% or higher identity to the amino acid sequence represented by any of SEQ ID NOs: 2, 4, 6, 8, 10, and 12, and having (i) the activity of converting active jasmonic acid to inactive jasmonic acid or (ii) the activity of enhancing the environmental stress tolerances of plants;

(d) a gene consisting of a nucleotide sequence represented by any of SEQ ID NOs: 1, 3, 5, 7, 9, and 11;

(e) a gene consisting of a polynucleotide having 90% or higher identity to the nucleotide sequence represented by any of SEQ ID NOs: 1, 3, 5, 7, 9, and 11, and encoding a protein having (i) the activity of converting active jasmonic acid to inactive jasmonic acid or (ii) the activity of enhancing the environmental stress tolerances of plants; and

(f) a gene consisting of a polynucleotide hybridizing under stringent conditions to a polynucleotide consisting of the nucleotide sequence represented by any of SEQ ID NOs: 1, 3, 5, 7, 9, and 11 or a nucleotide sequence complementary thereto, and encoding a protein having (i) the activity of converting active jasmonic acid to inactive jasmonic acid or (ii) the activity of enhancing the environmental stress tolerances of plants.

(25) A method for producing a plant, comprising the step of:

increasing the expression of one or two or more genes selected from a gene group consisting of a first gene subgroup consisting of Os12g0150200 gene, Os01g0858350 gene, Os05g0445100 gene, Os11g0151400 gene, AT3G48520 gene, and AT2G27690 gene, and a gene functionally equivalent to any of these genes and a second gene subgroup consisting of Os04g0584800 gene and a gene functionally equivalent to this gene, the one or two or more genes being endogenous or foreign genes.

(26) A method for preparing a plant having an enhanced environmental stress tolerance, comprising the step of increasing the expression of any gene selected from the group consisting of the following (a) to (f) in a plant:

(a) a gene encoding a protein comprising the amino acid sequence represented by SEQ ID NO: 26;

(b) a gene encoding a protein comprising an amino acid sequence derived from the amino acid sequence represented by SEQ ID NO: 26 by the deletion, substitution, addition, or insertion of one or more amino acids, and having the activity of enhancing the environmental stress tolerances of plants;

(c) a gene encoding a protein comprising an amino acid sequence having 90% or higher identity to the amino acid sequence represented by SEQ ID NO: 26, and having the activity of enhancing the environmental stress tolerances of plants;

(d) a gene consisting of the nucleotide sequence represented by SEQ ID NO: 25;

(e) a gene consisting of a polynucleotide having 90% or higher identity to the nucleotide sequence represented by SEQ ID NO: 25, and encoding a protein having the activity of enhancing the environmental stress tolerances of plants; and

(f) a gene consisting of a polynucleotide hybridizing under stringent conditions to a polynucleotide consisting of the nucleotide sequence represented by SEQ ID NO: 25 or a nucleotide sequence complementary thereto, and encoding a protein having the activity of enhancing the environmental stress tolerances of plants.

(27) A method for producing a plant, comprising the step of:

selecting a plant derived from mating by using, as an index, the expression of one or two or more genes selected from a gene group consisting of a first gene subgroup consisting of Os12g0150200 gene, Os01g0858350 gene, Os05g0445100 gene, Os11g0151400 gene, AT3G48520 gene, and AT2G27690 gene, and a gene functionally equivalent to any of these genes and a second gene subgroup consisting of Os04g0584800 gene and a gene functionally equivalent to this gene.

(28) A method for producing a crop, comprising the step of cultivating a crop which is a plant according to any of (1) to (18).
(29) A method for screening plants, comprising the steps of:

screening one or two or more plants by using, as an index, the expression of one or two or more genes selected from a gene group consisting of a first gene subgroup consisting of Os12g0150200 gene, Os01g0858350 gene, Os05g0445100 gene, Os11g0151400 gene, AT3G48520 gene, and AT2G27690 gene, and a gene functionally equivalent to any of these genes and a second gene subgroup consisting of Os04g0584800 gene and a gene functionally equivalent to this gene; and evaluating the salt tolerance of the plants having high expression levels of the one or two or more genes.

(30) A method for screening plants, comprising the steps of:

screening one or two or more plants by using, as an index, the expression of any gene selected from the group consisting of the following (a) to (f); and evaluating the salt tolerance of the plants having a high expression level of the gene:

(a) a gene encoding a protein comprising an amino acid sequence represented by any of SEQ ID NOs: 2, 4, 6, 8, 10, 12, and 26;

(b) a gene encoding a protein comprising an amino acid sequence derived from the amino acid sequence represented by any of SEQ ID NOs: 2, 4, 6, 8, 10, 12, and 26 by the deletion, substitution, addition, or insertion of one or more amino acids, and having the activity of enhancing the environmental stress tolerances of plants;

(c) a gene encoding a protein comprising an amino acid sequence having 90% or higher identity to the amino acid sequence represented by any of SEQ ID NOs: 2, 4, 6, 8, 10, 12, and 26, and having the activity of enhancing the environmental stress tolerances of plants;

(d) a gene consisting of a nucleotide sequence represented by any of SEQ ID NOs: 1, 3, 5, 7, 9, 11, and 25;

(e) a gene consisting of a polynucleotide having 90% or higher identity to the nucleotide sequence represented by any of SEQ ID NOs: 1, 3, 5, 7, 9, 11, and 25, and encoding a protein having the activity of enhancing the environmental stress tolerances of plants; and

(f) a gene consisting of a polynucleotide hybridizing under stringent conditions to a polynucleotide consisting of the nucleotide sequence represented by any of SEQ ID NOs: 1, 3, 5, 7, 9, 11, and 25 or a nucleotide sequence complementary thereto, and encoding a protein having the activity of enhancing the environmental stress tolerances of plants.

(31) Progeny, a descendent, or a clone of a plant according to any of (1) to (18).
(32) A propagating material derived from the progeny, the descendent, or the clone of the plant according to (31).
(33) The propagating material according to (32), wherein the propagating material is selected from the group consisting of a seed, a fruit, a cutting, a tuber, a tuberous root, a strain, a callus, and a protoplast.

The present specification encompasses the contents disclosed in U.S. Provisional Patent Application No. 62/275,697 on which the priority of the present application is based.

### BRIEF DESCRIPTION OF DRAWINGS

FIG. 1A is a diagram showing a molecular phylogenetic tree of Arabidopsis CYP94C1 (AT2G27690) and rice homologs (upper diagram) and amino acid sequence alignment (lower diagram).

FIG. 1B is a diagram showing the alignment of the amino acid sequences of Arabidopsis CYP94C1 (AT2G27690) and a rice homolog Os12g0150200.

FIG. 2A is a diagram showing results of evaluating the salt tolerance of various rice full-length cDNA-overexpressing (FOX) rice lines.

FIG. 2B is a diagram showing results of evaluating the salt tolerance of the wild type, a CYP94C2b FOX rice line (systematic name: FE047), and separately produced three CYP94C2b-overexpressing rice lines.

FIG. 2C is a diagram showing results of evaluating the salt tolerance (degrees of damage) of the wild type and the CYP94C2b-overexpressing line rice (FE047).

FIG. 2D is a diagram showing results of evaluating the salt tolerance (survival rates) of the wild type and the CYP94C2b-overexpressing line rice (FE047).

FIG. 2E is a diagram showing results of evaluating the salt tolerance (the number of seeds) of the wild type and the CYP94C2b-overexpressing line rice (FE047).

FIG. 2F is a diagram showing results of evaluating the salt tolerance (appearance) of the CYP94C2b-overexpressing line rice (FE047).

FIG. 3 FIG. 3 is a diagram showing change in the accumulations of jasmonic acid, active jasmonic acid, and inactive jasmonic acid induced after wounding.

FIG. 4A is a diagram showing the responses (shoot length) of the wild type and the FE047 line to jasmonic acid.

FIG. 4B is a diagram showing the responses (root elongation) of the wild type and the FE047 line to jasmonic acid.

FIG. 4C is a diagram showing the responses (shoot length) of the wild type and the FE047 line to coronatine (COR).

FIG. 4D is a diagram showing the responses (root elongation) of the wild type and the FE047 line to coronatine (COR).

FIG. 4E is a diagram showing the expression level of a JA-responsive gene (JAmyb) after wounding of the wild type and the FE047 line.

FIG. 4F is a diagram showing the expression level of a JA-responsive gene (JAZ11) after wounding of the wild type and the FE047 line.

FIG. 5A is a diagram showing results of evaluating the aging (senescence, appearance) of leaves induced in the wild type and the FE047 line by salt stress.

FIG. 5B is a diagram showing results of evaluating the degree of etiolation (senescence) of leaves induced in the wild type and the FE047 line by salt stress, on the basis of the colors of the leaves (green: indicated by dark gray, yellow-green: gray, yellow to brown: light gray).

FIG. 5C is a diagram showing results of evaluating the etiolation (senescence, upper diagram: aging stress marker gene (SGR gene), lower diagram: cytokinin-responsive marker gene (OsRR10 gene)) of leaves induced in the wild type and the FE047 line by salt stress.

FIG. 6A is a diagram showing results of evaluating the expression level of CYP94C2b in the wild type, a FE047 line (T2 generation), and a separately produced CYP94C2b-overexpressing rice line, and their salt tolerance. Viability indicates survival by + and death by −.

FIG. 6B is a diagram showing the results of evaluating the expression level of CYP94C2b in the wild type, the FE047 line (12), and an independent overexpressing line, and their salt tolerance (the relationship between the ranking of the expression level and survival rates).

FIG. 7 is a diagram showing results of identifying a rice gene to provide high salt tolerance by cDNA overexpression.

FIG. 8A is a diagram showing the provision of salt tolerance by the overexpression of Os12g0150200 gene and Os04g0584800 gene.

FIG. 8B is a diagram showing the provision of heat stress tolerance by the overexpression of Os12g0150200 gene and Os04g0584800 gene.

FIG. 8C is a diagram showing the provision of hyperosmotic stress tolerance and ion stress tolerance by the overexpression of Os04g0584800 gene.

FIG. 9 is a diagram showing results of a salt tolerance test on a standard rice variety (Nipponbare) and salt-tolerant rice varieties (Heitai and Pokkali) (A: in a laboratory and B: in a greenhouse) and the expression level of Os12g0150200 gene (C).

### DESCRIPTION OF EMBODIMENTS

The disclosure of the present specification relates to a gene to provide environmental stress tolerances by enhancement in its expression and the use thereof. The present disclosure is based on the successful identification of a gene that contributes to new salt stress tolerance by enhancement in its expression.

The present inventors focused on problems associated with evaluation approaches for evaluating environmental stress tolerances. Specifically, the present inventors searched for a gene capable of conferring an excellent environmental stress tolerance with higher accuracy at a practical level, on the basis of two types of parallel evaluation approaches. As a result, the present inventors found a gene that, surprisingly, does not exhibit a definite tolerance at an experimental level, but exerts higher or more diverse environmental stress tolerances at a practical level.

Any gene identified in the present disclosure was also found to be able to exert tolerances to two or more environmental stresses and to easily respond to combined environmental stresses that may occur in the natural environment. Use of such a gene allows two or more environmental stress tolerances to be provided by the high expression of one type of gene. Therefore, an appropriate stress tolerance gene can be selected and introduced in a plant sensitive to a plurality of stresses, and overexpressed to thereby develop a plant excellent in environmental stress tolerances.

Also, the present inventors confirmed that the expression level of the gene identified in the present disclosure, for example, a gene specified by Os12g0150200, was also enhanced under non-stressed conditions of existing salt-tolerant rice varieties. Specifically, the gene identified by the present inventors can develop a plant excellent in environmental stress tolerances such as salt tolerance by circumventing or suppressing adverse effects caused by enhancement in its expression on the plant.

In the present specification, the increased expression of a gene can include the increased expression level of the gene as well as an increased content of protein encoded by the gene (e.g., increase in protein level or improvement in the activity of the protein). Thus, a particular gene may be introduced as a foreign gene to increase the gene product. In addition, an expression control region, such as a promoter, of an endogenous gene is modified, and the expression of the endogenous gene may be increased by the expression control region.

Hereinafter, a gene to provide environmental stress tolerances, an expression vector, a plant, a method for producing a plant, a method for producing a crop, etc., in relation to the disclosure of the present specification will be described in order.

### (Gene Capable of Conferring Environmental Stress Tolerance)

The gene capable of conferring environmental stress tolerances is one or two or more genes selected from a gene group consisting of a first gene subgroup consisting of a gene specified by Os12g0150200 (in the present specification, simply referred to as Os12g0150200 gene), Os05g0445100 gene, Os11g0151400 gene, Os01g0858350 gene, AT3G48520 gene, and AT2G27690 gene, and a gene functionally equivalent to any of these genes and a second gene subgroup consisting of Os04g0584800 gene and a gene functionally equivalent to this gene. Hereinafter, the first gene subgroup and the second gene subgroup will be described in order.

### (First Gene Subgroup and Constituent Gene (First Gene))

The first gene can be selected from the first gene subgroup consisting of Os12g0150200 gene, Os05g0445100 gene, Os11g0151400 gene, Os01g0858350 gene, AT3G48520 gene, and AT2G27690 gene, and a gene functionally equivalent to any of these genes. The first gene subgroup encodes proteins having jasmonic acid-inactivating activity, as described below.

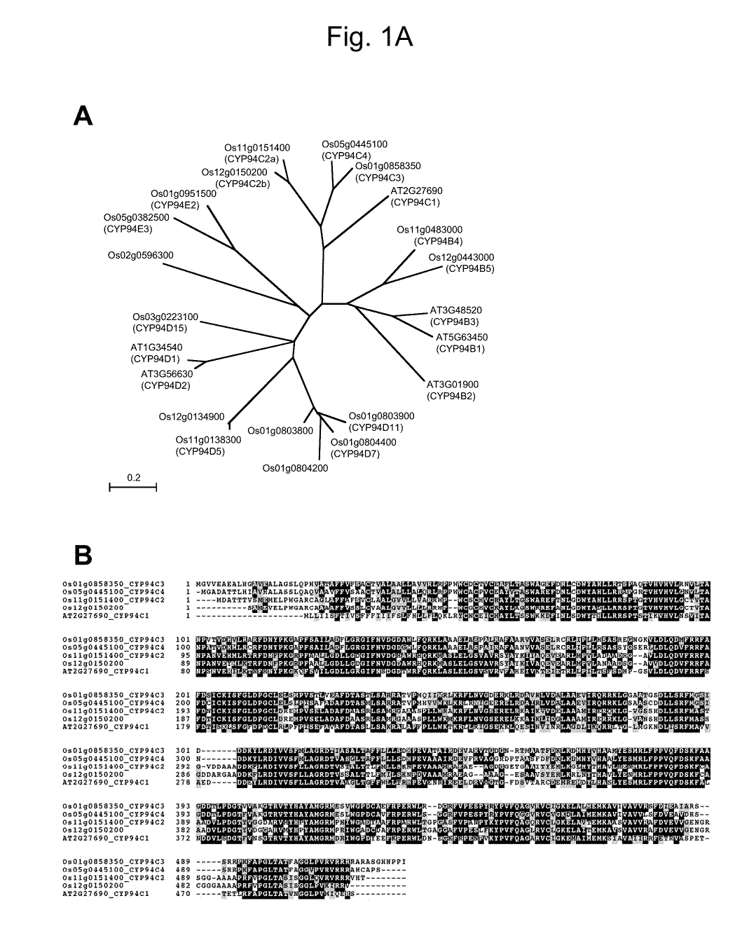

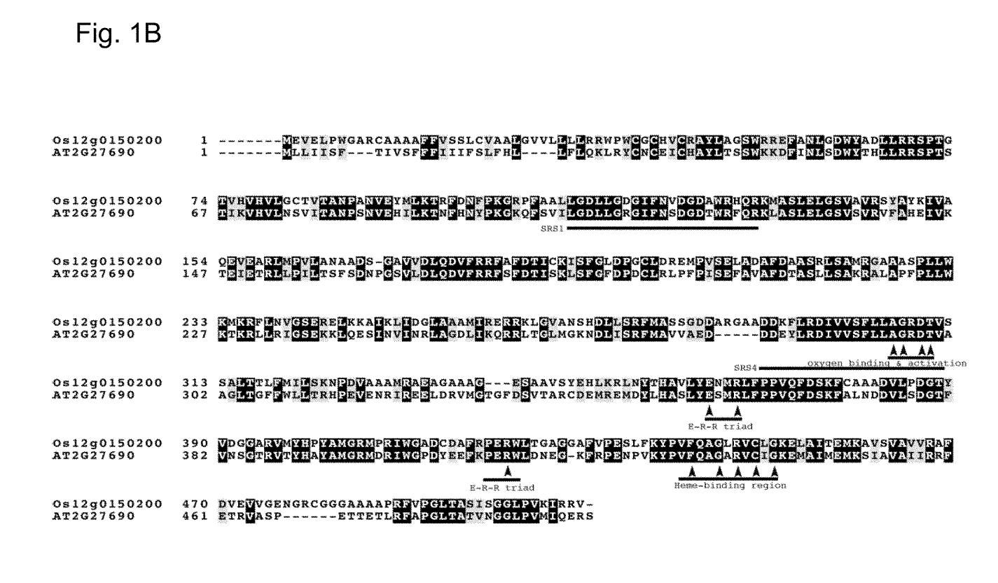

The respective gene products of the Os01g0858350 gene, the Os05g0445100 gene, the Os11g0151400 gene, the Os12g0150200 gene, the AT3G48520 gene, and the AT2G27690 gene are presumed to belong to the CYP94 family, which is the cytochrome P450 enzyme family. More specifically, as shown in the molecular phylogenetic tree and the alignment of FIG. 1, each of the Os01g0858350, Os05g0445100, Os11g0151400, and Os12g0150200 genes is considered to be Arabidopsis CYP94C1 protein, a protein encoded by AT2G27690, and its rice (Oryza saliva) homolog (ortholog). Also, Os01g0858350, Os05g0445100, Os11g0151400, and Os12g0150200 are considered to be homologs (paralogs) for each other.

The molecular phylogenetic tree and the alignment shown in FIG. 1 were obtained by performing the amino acid sequence comparison of proteins encoded by the Os01g0858350 gene, the Os05g0445100 gene, the Os11g0151400 gene, and the Os12g0150200 gene, and Arabidopsis CYP94C1 (AT2g27690) using CLUSTALW (Multiple Sequence Alignment; http://www.genome.jp/tools/clustalw/).

A protein encoded by AT3G48520 is also presumed to belong to the CYP94 family, which is the cytochrome P450 enzyme family.

The Arabidopsis CYP94C1 protein is considered to have the activity of introducing a hydroxy group to carbon at position 12 of active jasmonic acid, i.e., 7-isojasmonoylisoleucine, and converting 12-hydroxy-7-isojasmonoylisoleucine obtained as inactive jasmonic acid to 12-carboxyl-7-isojasmonoylisoleucine by oxidation (hereinafter, also referred to as jasmonic acid-inactivating activity) (Heitz, T., et al., 2012, J. Biol. Chem., 287: 6296-6306; Kitaoka, N., et al., 2011, Plant Cell Physiol., 52: 1757-1765; and Koo, A. J., et al., 2011, Proc. Natl. Acad. Sci. USA., 108: 9298-9303).

Also, the present inventors confirmed that, in transformants showing increased expression of the Os12g0150200 gene, jasmonic acid induced by wounding and its active form were decreased while its inactive form was increased. Furthermore, as shown in FIG. 1, according to the alignment of the amino acid sequence of AT2G27690 with the respective amino acid sequences of Os01g0858350, Os05g0445100, Os11g0151400, and Os12g0150200, all of substrate recognition site (SRS), heme-binding domain, ERR triad site, and oxygen-binding and activation sites in the Arabidopsis CYP94C1 protein are conserved. Thus, it is concluded that the proteins encoded by these genes also have jasmonic acid-inactivating activity.

It has already been known that the product of Arabidopsis AT3G48520 also has jasmonic acid-inactivating activity.

In conclusion, all of the first genes are considered to encode proteins (enzymes) having the activity of converting active jasmonic acid to inactive jasmonic acid.

The nucleotide sequences of the respective coding regions of the Os01g0858350 gene, the Os05g0445100 gene, the Os11g0151400 gene, and the Os12g0150200 gene among the first genes have 54% to 58% identity to the nucleotide sequence of the coding region of the AT2G27690 gene. Amino acid sequences encoded by the nucleotide sequences of the respective coding regions of the Os01g0858350 gene, the Os05g0445100 gene, the Os11g0151400 gene, and the Os2g0150200 gene among the first genes have 53% to 57% identity to an amino acid sequence encoded by the nucleotide sequence of the coding region of AT2G27690.

The first gene also encompasses a gene functionally equivalent to the gene thus identified. Such a functionally equivalent gene includes a homolog (paralog and ortholog) of the identified gene and can also encompass a gene encoding a protein having jasmonic acid-inactivating activity, irrespective of origin. It is preferred that the first gene should be derived mainly from the plant kingdom. The first gene may be derived from a dicotyledon including a plant of the family Leguminosae, such as soybean, or a monocotyledon including a plant of the family Poaceae, such as rice, corn, or sugarcane.

The gene functionally equivalent to a particular gene can be obtained, for example, by searching a database such as NCBI (National Center for Biotechnology Information; http://www.ncbi.nlm.nih.gov) with the nucleotide sequence of the gene or the amino acid sequence of a protein encoded by the gene as a query sequence and evaluating a gene having high identity for whether to be functionally equivalent to the gene, for example, whether or not the gene encodes a protein (enzyme) having the activity of converting active jasmonic acid to inactive jasmonic acid in the present disclosure.

The first gene is appropriately selected according to a plant to be transformed. Examples of the soybean gene functionally equivalent to the Os12g050200 gene include a gene encoding cytochrome P450 94A1-like [Glycine max] (accession Nos., nucleotide sequence: XM_006592251.1 (SEQ ID NO: 13), amino acid sequence: XP_006592314.1 (SEQ ID NO: 14)), a gene encoding cytochrome P450 94A2-like [Glycine max] (accession Nos., nucleotide sequence: XM_003538086.2 (SEQ ID NO: 15), amino acid sequence: XP 003538134.1 (SEQ ID NO: 16)), and a gene encoding cytochrome P450 94A1-like [Glycine max] (accession Nos., nucleotide sequence: XM_006577004.1 (SEQ ID NO: 17), amino acid sequence: XP_006577067.1 (SEQ ID NO: 18)). The identities of these three genes to the amino acid sequence of the protein encoded by the Os12g0150200 gene and the amino acid sequence of the Arabidopsis CYP94C1 protein (protein encoded by the AT2G27690 gene) were 56.8%/63.4%, 57.8%/59.9%, and 53.8%/57.8%, respectively.

Examples of the corn gene functionally equivalent to the Os12g0150200 gene include a gene encoding a functionally unknown protein (accession Nos., nucleotide sequence: BT086294.1 (SEQ ID NO: 19), amino acid sequence: ACR36647.1 (SEQ ID NO: 20)), a gene encoding cytochrome P450 CYP94C20 (accession Nos., nucleotide sequence: EU956091.1 (SEQ ID NO: 21), amino acid sequence: ACG28209.1 (SEQ ID NO: 22)), and a gene encoding cytochrome P450 CYP94D27 (accession Nos., nucleotide sequence: EU975752.1 (SEQ ID NO: 23), amino acid sequence: ACG47870.1 (SEQ ID NO: 24)). The identities of these three genes to the amino acid sequence of the protein encoded by the Os12g0150200 gene and the amino acid sequence of the Arabidopsis CYP94C1 protein (protein encoded by the AT2G27690 gene) were 81.2%/53.9%, 82.1%/54.3%, and 43.8%/42.6%, respectively.

The first gene may be naturally prepared or may be artificially prepared as long as the first gene encodes the protein having jasmonic acid-inactivating activity as described above. Thus, in addition to various genes described above, these genes having an artificially introduced variation may be used. Also, the gene may be genomic DNA or may be cDNA or the like.

The first gene may be specified by the coding region of each gene and/or a protein having an amino acid sequence encoded by the coding region. As for the protein encoded by the first gene, those skilled in the art can appropriately gain information on such a gene and a protein by making access to HP of NCBI (National Center for Biotechnology Information; http://www.ncbi.nlm.nih.gov), etc. It is preferred that the protein encoded by the first gene should be derived mainly from the plant kingdom. The protein may be derived from a dicotyledon or a monocotyledon and may be particularly derived from a plant of the family Poaceae. Those skilled in the art can appropriately gain information on such a gene and a protein by making access to HP of NCBI (National Center for Biotechnology Information; http://www.ncbi.nlm.nih.gov), etc. Hereinafter, the protein encoded by the first gene (hereinafter, also referred to as a first protein) will be described.

In one embodiment, examples of the first protein include a protein comprising the amino acid sequence represented by SEQ ID NO: 2 encoded by the Os12g0150200 gene. In another embodiment, in addition to SEQ ID NO: 2, the present protein may be a protein having a given relation to known sequence information such as SEQ ID NO: 1 (nucleotide sequence of the coding region of the Os12g0150200 gene) or 2 as long as the protein has jasmonic acid-inactivating activity.

In an alternative embodiment, examples of the first protein include a protein having an amino acid sequence derived from the amino acid sequence represented by SEQ ID NO: 2 by the substitution, deletion, addition, and/or insertion of one or more amino acids, and having jasmonic acid-inactivating activity.

The amino acid sequence represented by SEQ ID NO: 2 (Os12g0150200) has two substrate recognition sites (SRS1 (21 amino acids) and SRS4 (20 amino acids)) as shown in FIG. 1B.

The jasmonic acid-inactivating activity can be obtained by detecting catalytic activity against reaction to form inactive jasmonic acid with active jasmonic acid as a substrate. The phrase “having jasmonic acid-inactivating activity” is not limited by its degree as long as having this activity.

The amino acid variation in the amino acid sequence represented by SEQ ID NO: 2 may be any one type of variation, i.e., deletion, substitution, addition, or insertion, or two or more types thereof may be combined. The total number of these variations is not particularly limited and is on the order of preferably 1 or more and 20 or less, more preferably 1 or more and 10 or less. The total number thereof is even more preferably 1 or more and 5 or less, further preferably 1 or more and 4 or less, still further preferably 1 or more and 3 or less.

Preferred examples of the amino acid substitution include conservative substitution and specifically include substitution within the following group: (glycine and alanine), (valine, isoleucine, and leucine), (aspartic acid and glutamic acid), (asparagine and glutamine), (serine and threonine), (lysine and arginine), or (phenylalanine and tyrosine).

It is preferred that the variation in the amino acid sequence represented by SEQ ID NO: 2 should be present at a site other than the substrate recognition sites in the amino acid sequence represented by SEQ ID NO: 2. In other words, it is preferred that even a variant should have high identity, at the two substrate recognition sites, to AT2G27690. Specifically, the identity of the amino acid sequence at each substrate recognition site in FIG. 1B is preferably 80% or higher, more preferably 85% or higher, further preferably 90% or higher, still further preferably 95% or higher. In the amino acid sequence moieties (17 amino acids for SRS1, 18 amino acids for SRS4) represented by identical amino acid residues between Os12g0150200 and AT2G27690 at each substrate recognition site, preferably 1 to 4 or less, more preferably 1 to 2 or less amino acids are based on conservative substitution, and further preferably, all of these amino acids are identical therebetween.

In an alternative embodiment, examples of the first protein include a protein having an amino acid sequence having 60% or higher identity to the amino acid sequence represented by SEQ ID NO: 2, and having jasmonic acid-inactivating activity. The identity is preferably 70% or higher, more preferably 80% or higher, even more preferably 85% or higher, further preferably 90% or higher, still further preferably 95% or higher, still further preferably 98% or higher.

In the present specification, the identity or similarity is, as known in the art, the relationship between two or more proteins or two or more polynucleotides determined by comparing their sequences. The “identity” in the art means the degree of sequence invariability between protein or polynucleotide sequences as determined by the alignment between the protein or polynucleotide sequences or, in some cases, by the alignment between series of such sequences. The similarity means the degree of correlation between protein or polynucleotide sequences as determined by the alignment between the protein or polynucleotide sequences or, in some cases, by the alignments between series of partial sequences. More specifically, the similarity is determined by sequence identity and conservation (substitution to maintain a particular amino acid or sequence in a sequence or the physicochemical properties of the sequence). The similarity is indicated by Similarity in results of sequence homology search using BLAST mentioned later. It is preferred that a method for determining the identity and the similarity should be a method designed to attain the longest alignment between sequences to be compared. The method for determining the identity and the similarity is provided as a publicly available program. For example, the BLAST (basic local alignment search tool) program by Altschul et al. (e.g., Altschul S. F., et al., 1990, J. Mol. Biol., 215: 403-410; and Altschul S. F., et al., 1997, Nucleic Acids Res., 25: 3389-3402) can be used in the determination. Conditions for using software such as BLAST are not particularly limited, and it is preferred to use default values.

For a nucleotide sequence encoding the amino acid sequence represented by SEQ ID NO: 2 or an amino acid sequence having a given relation as described above to the amino acid sequence, at least one base in a nucleotide sequence encoding a predetermined amino acid sequence can be substituted by a different type of base without changing the amino acid sequence of the protein, according to the degeneracy of the genetic code. Thus, the present gene also encompasses a gene having a nucleotide sequence altered by substitution based on the degeneracy of the genetic code.

The first protein is also a protein encoded by a polynucleotide comprising the nucleotide sequence represented by SEQ ID NO: 1. In a further alternative embodiment, examples thereof include a protein encoded by a polynucleotide hybridizing under stringent conditions to a polynucleotide consisting of a nucleotide sequence complementary to a polynucleotide consisting of the nucleotide sequence represented by SEQ ID NO: 1, and having jasmonic acid-inactivating activity.

The stringent conditions refer to, for example, conditions under which a so-called specific hybrid is formed while a nonspecific hybrid is not formed. Examples thereof include conditions under which hybridization takes place with a complementary strand of a nucleic acid having high nucleotide sequence identity, i.e., DNA consisting of a nucleotide sequence having 60% or higher, preferably 70% or higher, more preferably 80% or higher, further preferably 85% or higher, still further preferably 90% or higher, still further preferably 95% or higher, most preferably 98% or higher identity to the nucleotide sequence represented by SEQ ID NO: 1, but does not take place with a complementary strand of a nucleic acid having lower homology. More specifically, the stringent conditions refer to conditions involving a sodium salt concentration of 15 to 750 mM, preferably 50 to 750 mM, more preferably 300 to 750 mM, a temperature of 25 to 70° C., preferably 50 to 70° C., more preferably 55 to 65° C., and a formamide concentration of 0 to 50%, preferably 20 to 50%, more preferably 35 to 45%. In addition, for the stringent conditions, filter washing conditions after hybridization usually involve a sodium salt concentration of 15 to 600 mM, preferably 50 to 600 mM, more preferably 300 to 600 mM, and a temperature of 50 to 70° C., preferably 55 to 70° C., more preferably 60 to 65° C.

More specific examples of the stringent conditions include hybridization at 45° C. in 6×SSC (sodium chloride/sodium citrate) followed by washing with 0.2 to 1×SSC and 0.1% SDS at 50 to 65° C. Alternatively, examples of such conditions can include hybridization at 65 to 70° C. in 1×SSC followed by washing with 0.3×SSC at 65 to 70° C. The hybridization can be performed by a conventionally known method such as a method described in J. Sambrook et al., Molecular Cloning: A Laboratory Manual 2nd Ed., Cold Spring Harbor Laboratory Press, 10 Skyline Drive Plainview, N.Y. (1989).

In light of those described above, in a further alternative embodiment, examples thereof include a protein encoded by a polynucleotide having a nucleotide sequence having 70% or higher, preferably 80% or higher, more preferably 85% or higher, further preferably 90% or higher, still further preferably 95% or higher, most preferably 98% or higher identity to the nucleotide sequence represented by SEQ ID NO: 1, and having jasmonic acid-inactivating activity.

Various embodiments of the first protein in the same forms as those described about Os12g0150200 are also applied to other first genes, i.e., the Os05g0445100 gene, the Os11g0151400 gene, the Os01g0858350 gene, the AT3G48520 gene, and the AT2G27690 gene, etc. Further embodiments of the first gene are thereby specified. The nucleotide sequences of the respective coding regions of the Os05g0445100 gene, the Os11g0151400 gene, the Os01g0858350 gene, the AT3G48520 gene, and the AT2G27690 gene, and the amino acid sequences thereof are represented by SEQ ID NOs: 3 and 4, SEQ ID NOs: 5 and 6, SEQ ID NOs: 7 and 8, SEQ ID NOs: 9 and 10, and SEQ ID NOs: 11 and 12, respectively.

The first gene encoding the first protein in various embodiments described above can be obtained as a nucleic acid fragment, for example, by performing PCR amplification using primers designed on the basis of the sequence of SEQ ID NO: 1 or the like and DNA extracted from a plant of the family Poaceae or the like, or a nucleic acid derived from various cDNA libraries or genomic DNA libraries or the like as a template. Also, the first gene can be obtained as a nucleic acid fragment by performing hybridization using the nucleic acid derived from libraries or the like as a template and a DNA fragment, which is a portion of the present gene, as a probe. Alternatively, the present gene may be synthesized as a nucleic acid fragment by various nucleic acid sequence synthesis methods known in the art, such as a chemical synthesis method.

The first gene encoding the first protein in various embodiments described above can be obtained, for example, by modifying DNA encoding the amino acid sequence represented by SEQ ID NO: 2 (e.g., the DNA consists of the nucleotide sequence represented by SEQ ID NO: 1) by mutagenesis, site-directed mutagenesis, a molecular evolutionary approach using error-prone PCR, or the like routinely used. Examples of such an approach include known approaches such as the Kunkel method and the gapped duplex method, and methods equivalent thereto. A variation is introduced using, for example, a kit for variation introduction using site-directed mutagenesis (e.g., Mutant-K (manufactured by Takara Bio Inc.) or Mutant-G (manufactured by Takara Bio Inc.)) or using LA PCR in vitro Mutagenesis series kit from Takara Bio Inc.

In addition, those skilled in the art can obtain the first gene encoding the first protein in various embodiments on the basis of, for example, the known sequence of SEQ ID NO: 1 or 2, with reference to Sambrook J. et al., Molecular Cloning (1989) (supra) or the like.

### (Second Gene Subgroup and Constituent Gene (Second Gene))

The second gene can be selected from the second gene subgroup consisting of Os04g0584800 gene and a gene functionally equivalent to this gene. A protein encoded by the Os04g0584800 gene is functionally unknown, but is a protein having a RAS/GTP-binding domain at the N-terminal side and an adaptin-binding domain (SEQ ID NO: 27) at the C-terminal side. Examples of the gene functionally equivalent to the Os04g0584800 gene include a gene encoding a protein having a RAS/GTP-binding domain at the N-terminal side and an adaptin-binding domain at the C-terminal side, and having predetermined or higher identity to the amino acid sequence of the Os04g0584800 gene.

In addition to the homolog described above, genes having an artificially introduced variation may be used. The gene may be genomic DNA or may be cDNA or the like.

The gene functionally equivalent to the Os04g0584800 gene is a gene that can enhance the tolerance of a plant to salt stress when the expression of the gene is increased. In other words, the gene functionally equivalent thereto is a gene encoding a protein having the activity of enhancing the tolerance of a plant to salt stress when the expression of the gene is increased.

Examples of the gene functionally equivalent to the Os04g0584800 gene include Arabidopsis AT5G65960 gene, common grape LOC100266179 gene, cottonwood (one kind of poplar) POPTR_0002s17770g gene, and soybean LOC100813911 [Glycine max] gene. Further examples thereof include a gene encoding a barley predicted protein [Hordeum vulgare subsp. vulgare].

The nucleotide sequence of the coding region of the barley predicted protein-encoding gene serving as the second gene has 83% identity to the nucleotide sequence of the coding region of Os04g0584800. Monocotyledons contain the nucleotide sequence having such high identity, whereas, genes of the other plant species exhibit 57 to 64% identity to the nucleotide sequence of the coding region of Os04g0584800. Amino acid sequences encoded by the nucleotide sequences of coding regions contained in these genes have 48% to 77% identity to an amino acid sequence encoded by the nucleotide sequence of the coding region of Os04g0584800.

The second gene may be specified by the coding region of each gene and/or a protein having an amino acid sequence encoded by the coding region, as with the first gene. The second gene may be naturally prepared or may be artificially prepared as long as the second gene encodes the protein having the activity of enhancing the tolerance to salt stress when expressed in a plant.

The nucleotide sequence of the coding region of the Os04g0584800 gene as the second gene, and the amino acid sequence of the protein encoded thereby are represented by SEQ ID NO: 25 (NM_001060207) and SEQ ID NO: 26 (NP_001053672), respectively. Likewise, the nucleotide sequence and the amino acid sequence are represented by SEQ ID NOs: 28 and 29 (NM_125993 and NP_569023), respectively, for the Arabidopsis AT5G65960, SEQ ID NOs: 30 and 31 (XP_002276437 and XM_002276401), respectively, for the common grape LOC100266179, SEQ ID NOs: 32 and 33 (XP_002302663 and XM_002302627), respectively, for the cottonwood (one kind of poplar) POPTR_0002s17770g, and SEQ ID NOs: 34 and 35 (BAJ96675 and AK365472), respectively, for the gene encoding a barley predicted protein [Hordeum vulgare subsp. vulgare] and represented by SEQ ID NOs: 36 and 37 (XP 003534231 and XM_003534183), respectively, for the soybean LOC100813911 [Glycine max].

Various embodiments described about the first gene can be adopted to the second gene according to the protein encoded by the second gene.

In the present specification, the “activity of enhancing the tolerance to salt stress when increased in a plant” refers to, for example, the activity of enhancing at least the tolerance to salt stress as compared with a wild-type plant when the second gene is expressed in the wild-type plant, as disclosed in Examples. In this context, the wild-type plant can typically be O. sativa Nipponbare. The second gene can be introduced in a plant via Agrobacterium to enhance its expression. As promoters for controlling the expression of the first and second genes, it is preferred to use constituent expression promoters. For the first gene, it is preferred to appropriately adjust the expression level according to the need. The tolerance to salt stress can typically be evaluated by use of an evaluation approach disclosed in Examples as evaluation at a practical level.

For example, for the first gene such as the Os12g0150200 gene, the expression level can be increased to 5 or more times to 150 or less times the expression level in the wild type (which corresponds to, for example, Nipponbare for rice) of the plant. This expression level may be 10 or more times, may be 20 or more times, or may be 30 or more times. Also, this expression level may be 100 or less times, may be 80 or less times, may be 70 or less times, may be 60 or less times, or may be 50 or less times. The expression level can be evaluated from a known approach, for example, the level of mRNA which is an expression product of the first gene.

It is preferred that the protein encoded by the second gene should be derived mainly from the plant kingdom, as with the first gene. The protein may be derived from a dicotyledon or a monocotyledon and may be particularly derived from a plant of the family Poaceae. Those skilled in the art can appropriately gain information on such a gene and a protein by making access to HP of NCBI (National Center for Biotechnology Information; http://www.ncbi.nlm.nih.gov), etc.

The first gene and/or the second gene can be used for conferring or enhancing environmental stress tolerances to a plant. Only the first gene may be used, or only the second gene may be used. One or two or more first genes can be used, and one or two or more second genes can also be used.

The first gene can provide a favorable tolerance to salt stress (hereinafter, a good or poor stress tolerance will be described with respect to wild-type O. sativa Nipponbare (Japanese rice)). Particularly, the first gene can provide a high tolerance to salt stress. Thus, it is preferred to apply the first gene to a plant predicted to undergo a high level of salt stress. Furthermore, the first gene can provide a favorable tolerance to heat stress. Thus, it is preferred that the first gene should be applied to a plant placed under an environment predicted to have both of salt stress and heat stress.

On the other hand, the second gene can provide a favorable tolerance to salt stress. The second gene can further provide a favorable tolerance to ion stress. Thus, it is preferred that the second gene should be applied to a plant predicted to undergo salt stress and ion stress. Moreover, the second gene can also provide a favorable tolerance to heat stress and/or hyperosmotic stress. Thus, it is preferred that the second gene should be applied to a plant under an environment predicted to have heat stress and/or hyperosmotic stress in addition to salt stress and/or ion stress.

In the present specification, the salt stress refers to salt stress that is evaluated by a saline stress tolerance test (closed greenhouse) similar to the salt tolerance test of International Rice Research Institute (IRRI). Specifically, the salt stress refers to salt stress that is evaluated by conducting the following hydroponic cultivation test on the basis of the description of Thomson M. L., et al., 2010, Characterizing the Saltol Quantitative Trait Locus for Salinity Tolerance in Rice, Rice 3: 148-160. A germinated young plant is put in a float with a net, grown in pure water for 3 days, and then further hydroponically cultivated in a Yoshida medium. The electrical conductivity (EC) of the hydroponic medium is adjusted to 6 dSm⁻¹ for the first 3 days and then set to 12 dSm⁻¹ by the dissolution of NaCl. In this respect, the EC and pH of the medium are adjusted by the addition of water or NaOH every 2 to 3 days. After 2 weeks, signs caused by salt stress are evaluated using the evaluation score of Thomson et al. (2010) (supra).

In the present specification, the hyperosmotic stress can be evaluated on the basis of a survival rate when a plant hydroponically cultivated for 3 days in pure water and for 15 days in a Yoshida medium is placed for 7 days in a medium containing 26% polyethylene glycol (PEG4000) and for 4 days in a medium free from PEG4000 (or the survival rate may be compared with the survival rate of a wild type under the same conditions as above).

In the present specification, the heat stress can be evaluated on the basis of a survival rate when a plant hydroponically cultivated at 28° C. for 3 days in pure water and for 17 days in a Yoshida medium is placed at 42° C. for 7 days, brought back to 28° C. and placed at this temperature for 7 days (or the survival rate may be compared with the survival rate of a wild type under the same conditions as above).

In the present specification, the ion stress can be evaluated on the basis of a survival rate when a 4-cm-long aerial part is cut out of a plant grown for 7 days in a Murashige-Skoog (MS) medium, transplanted to a MS medium containing 40 mM LiCl, and grown for 2 weeks (or the survival rate may be compared with the survival rate of a wild type under the same conditions as above).

The first gene and the second gene can both provide a tolerance to salt stress and can further provide tolerances to additional environmental stresses, by enhancement in their expression in a plant.

### (Expression Vector)

The expression vector disclosed in the present specification can be an expression vector for expressing the first gene and/or the second gene in plant cells. The vector of the present disclosure can have the first gene and/or the second gene. The present vector can be intended to introduce the present gene as foreign DNA and consequently enhance the expression of the present gene, regardless of the presence or absence of the endogenous present gene on the chromosomes in host cells (plant cells). The case is not excluded where the present vector is intended to enhance the expression of the endogenous present gene on the chromosomes in plant cells by homologous recombination or the like.

Examples of the plant cells include, but are not particularly limited to, cells of Arabidopsis, soybean, rice, corn, potato, and tobacco. A monocotyledon is preferred, and a plant of the family Poaceae is more preferred. Examples of the plant of the family Poaceae include rice, wheat, barley, corn, sorghum, and sugarcane. The plant cells also include cultured cells such as suspension-cultured cells as well as protoplasts and calluses. The plant cells also include cells in plants such as sections of leaves in addition to shoot primordia, multiple shoots, hairy roots, and the like.

When the present vector is intended to introduce and express the present gene as foreign DNA in plant cells, the present vector can have a promoter that permits transcription in plant cells, and the present gene operably linked under the control of the promoter. The present vector can further comprise a terminator containing a poly A addition signal. Examples of such a promoter include a promoter for constitutively or inductively expressing the present gene. Examples of the promoter for constitutive expression include cauliflower mosaic virus 35S RNA promoter (Odell, et al., 1985, Nature, 313: 810), rice actin 1 gene promoter (Zhang, et al., 1991, Plant Cell, 3: 1155), and corn polyubiquitin 1 gene promoter (Cornejo, et al., 1993, Plant Mol. Biol., 23: 567). Examples of the promoter for inductively expressing the present gene include a promoter of a gene known to be expressed by external causes such as mycotic, bacterial, or viral infection or invasion, low temperatures, high temperatures, dryness, ultraviolet irradiation, and spray of particular compounds. Examples of such a promoter include rice chitinase gene promoter (Xu, et al., 1996, Plant Mol. Biol., 30: 387), tobacco PR protein gene promoter (Ohshima, et al., 1990, Plant Cell, 2: 95), rice “lip19” gene promoter (Aguan, et al., 1993, Mol. Gen. Genet., 240: 1), rice “hsp80” gene and “hsp72” gene promoters (Van Breusegem et al., 1994, Planta, 193: 57), Arabidopsis “rab16” gene promoter (Mundy, et al., 1990, Proc. Natl. Acad. Sci. USA, 87: 1406), parsley chalcone synthase gene promoter (Schulze-Lefert, et al., 1989, EMBO J., 8:651), and maize (corn) alcohol dehydrogenase gene promoter (Walker, et al., 1987, Proc. Natl. Acad. Sci. USA, 84: 6624).

The present vector may be intended to allow host cells of cells such as E. coli, yeast, animal or plant cells, or insect cells to produce the first protein and/or the second protein as a recombinant protein. In this case, the present vector can have the present gene under the control of a promoter operable in appropriate host cells.

Those skilled in the art can construct the present vector, for example, using a commercially available material such as various plasmids known to those skilled in the art. The present vector can be constructed using, for example, plasmids “pB1121”. “pB1221”, and “pB1101” (all manufactured by Clontech Laboratories, Inc.) as well as vectors for expressing the present gene in plant cells for the preparation of a transformed plant.

According to the disclosure of the present specification, a host cell, such as a plant cell, having such an expression vector introduced therein is also provided. Also, a drug for improving the environmental stress tolerances of a plant, comprising the first gene and/or the second gene as an active ingredient, can also be provided. The present drug may comprise the present vector as the first gene and/or the second gene as an active ingredient.

### (Plant)

The plant disclosed in the present specification encompasses a transformed plant as well as a plant derived from mating or mutation. The plant disclosed in the present specification has the increased expression of the first gene and/or the second gene. In the transformed plant, the first gene and/or the second gene to be expressed differs depending on the form of the plant and may be an endogenous gene in the plant or may be a foreign gene. Expression of both of these may be increased.

The increased expression of the gene means that the expression level of the gene (the amount of a primary transcription product of the gene as well as the amount of a produced protein encoded by the gene) has been increased as compared with that before transformation or the activity of the protein has been increased as compared with that before transformation. As a result of increasing the expression of the gene, the expression level of the gene may be increased while the activity itself of the present protein may be increased. The expression levels of these genes can be evaluated by a known method, i.e., by using the amounts of transcription products or translation products of these genes as an index.

### (Transformed Plant)

The transformed plant disclosed in the present specification has the increased expression of the first gene and/or the second gene. In the transformed plant, the first gene and/or the second gene to be expressed may be an endogenous gene in the plant or may be a foreign gene. Expression of both of these may be increased.

The form of the increased expression of the gene in the transformed plant is not particularly limited. Examples thereof include a form in which a promoter operable in plant cells and the present gene operably linked to the promoter are intrachromosomally or extrachromosomally retained as foreign DNA in the plant cells. The gene to be linked to the promoter may be an endogenous gene in the plant cells or may be a foreign gene. In order to improve the activity of the promoter of the endogenous present gene, further examples thereof include a form in which the whole or a portion of the promoter region on the chromosomes is substituted, and a form in which the promoter region is substituted together with the endogenous gene.

The transformed plant typically includes a plant cell having introduced therein the vector disclosed in the present specification which is intended to introduce and express the gene in the plant cell.

The present transformed plant can be obtained by regenerating a plant from the plant cell transformed by the introduction of the vector disclosed in the present specification.

Various methods known to those skilled in the art, such as a polyethylene glycol method, electroporation, an Agrobacterium-mediated method, and a particle gun method can be used in the introduction of the vector in plant cells. Examples thereof include various methods such as gene transfer into protoplasts via polyethylene glycol (Datta, S. K., 1995, In Gene Transfer To Plants (Potrykus, I. and Spangenberg, G. Eds.) pp 66-74), gene transfer into protoplasts under electrical pulses (Toki, et al., 1992, Plant Physiol., 100: 1503-1507), direct introduction of genes in cells by the particle gun method (Christou, et al., 1991, Bio/Technology, 9: 957-962), and Agrobacterium-mediated introduction of genes (Hiei, et al., 1994, Plant J., 6: 271-282; and Toki, et al., 2006, Plant J., 47: 969-976). It is possible to perform the regeneration of a plant from the transformed plant cell by a method known to those skilled in the art according to the type of the plant cell (see Toki, et al., 1992. Plant Physiol., 100: 1503-1507). Examples thereof include: for rice, the method of Fujimura et al. (Plant Tissue Culture Lett., 2: 74); for corn, the methods of Shillito et al. (1989, Bio/Technology, 7: 581) and Gorden-Kamm et al. (1990, Plant Cell, 2: 603); for potato, the method of Visser et al. (1989, Theor. Appl. Genet., 78: 594); for tobacco, the method of Nagata and Takebe (1971, Planta, 99: 12); and for Arabidopsis, the method of Akama et al. (1992, Plant Cell Rep., 12: 7-11).

It is possible to perform the regeneration of a plant from the transformed plant cell by a method known to those skilled in the art according to the type of the plant cell (see Toki, et al., 1992, Plant Physiol., 100: 1503-1507). For example, as approaches of producing a transformed plant of rice, several techniques such as a method of gene-transfecting protoplasts using polyethylene glycol (PEG) and regenerating a plant (Indian rice varieties are suitable) (Datta, S. K., 1995, in Gene Transfer To Plants (Potrykus, I. and Spangenberg, G. Eds.) pp 66-74), a method of gene-transfecting protoplasts by electrical pulses and regenerating a plant (Japanese rice varieties are suitable) (Toki, et al., 1992, Plant Physiol., 100: 1503-1507), a method of directly introducing genes to cells by the particle gun method and regenerating a plant (Christou, et al., 1991, Bio/technology, 9: 957-962), and a method of introducing genes via Agrobacterium and regenerating a plant (Hiei, et al., 1994, Plant J., 6: 271-282; and Toki, et al., 2006, Plant J., 47: 969-976) have already been established and widely used in the technical field of the invention of the present application. In the present invention, these methods can be suitably used.

If a transformed plant having the present gene integrated on the genome is obtained, it is possible to obtain a descendent from the plant by sexual reproduction or asexual reproduction. It is also possible to obtain a propagating material (e.g., a seed, a fruit, a cutting, a tuber, a tuberous root, a strain, a callus, or a protoplast) from the plant or its descendent or clone, followed by the mass production of the plant on the basis thereof. The disclosure of the present specification includes (1) the plant cell having the present gene introduced therein and (2) a plant comprising the cell as already described as well as (3) a descendent and a clone of the plant, and (4) a propagating material from any of the plant and its descendent and clone.

The first gene and the second gene for use in transformation can be derived from various origins. The origins are not limited to a plant of the same species as the plant to be transformed, and application beyond classification is also possible. However, in the case where the target plant is a monocotyledon, a monocotyledon-derived gene is preferred, and a gene of the same family and the same species thereas is more preferred. The same holds true for a dicotyledon.

(Plant Derived from Mating or Mutation)

An alternative form of the plant disclosed in the present specification is a plant derived from mating or mutation. The plant derived from mating can retain the first gene and/or the second gene at the intrinsic gene loci of these genes on the chromosomes or at positions corresponding to the gene loci. In this context, the intrinsic gene locus of the gene on the chromosomes is a gene locus on the chromosomes where the gene resides in the case where the plant originally has the present gene or a homolog thereof. The position corresponding to the intrinsic gene locus on the chromosomes refers to a position that does not perfectly agree with the gene locus, but is close to the gene locus from nucleotide sequences upstream and downstream of the gene without interfering with the activity of increasing the expression of the present gene of the second DNA. It is preferred that such a plant should be a plant preliminarily having the endogenous present gene on the chromosomes.

In general, a plant having the increased expression of the first gene and/or the second gene by mating or mutation can be obtained in a breeding process such as mating or mutation by, for example, a variation, such as polymorphism caused by mutation, in the first gene and/or the second gene, or an epigenetic variation (including methylation) in a coding region or a control region. Alternatively, the plant can be obtained by using such a parent plant (which may be the transformant disclosed in the present specification).

The plant having the increased expression of the first gene and/or the second gene can be obtained by screening plants in a usual breeding process by using the expression level of the first gene and/or the second gene as an index, and further evaluating the environmental stress tolerances of a selected plant. Whether a plant has the first gene and/or the second gene in the breeding process can be confirmed by detecting the presence or absence of an amplification product, the length thereof, etc., by PCR on a DNA region containing the gene. Also, whether such a DNA region is present at the gene locus can be confirmed by a known nucleotide sequence determination method.

For example, according to mating, a F1 generation that retains the first gene and/or the second gene at the intrinsic positions of these genes on the chromosomes or at positions corresponding to these positions can be obtained, for example, by homologous recombination during the fertilization of a parent plant having the increased expression of the first gene and/or the second gene with another plant. A homozygote as to alleles of the first gene and/or the second gene can be obtained by using a F2 generation, back crossing, etc. The plant may be a heterozygote as to alleles containing the present DNA region as to the first and/or second gene and is preferably a homozygote.

The plant to which the first gene and/or the second gene disclosed in the present specification is applied, in other words, the plant whose environmental stress tolerances are improved, is not particularly limited. Specifically, the environmental stress tolerance-improving effect on every plant can be expected by increasing the expression of the gene.

Examples of the target plant include dicotyledons and monocotyledons, for example, plants belonging to the families Brassicaceae, Poaceae, Solanaceae, Leguminosae, and Salicaceae (see below), though not limited to these plants.

Brassicaceae: thale-cress or Arabidopsis (Arabidopsis thaliana), rapeseed (Brassica rapa, Brassica napus, and Brassica campestris), cabbage (Brassica oleracea var. capitata), Chinese cabbage (Brassica rapa var. pekinensis), Green pak choi or pak choi (Brassica rapa var. chinensis), turnip (Brassica rapa var. rapa), Nozawana (Brassica rapa var. hakabura), Mizuna (Brassica rapa var. laciniifolia), Komatsuna (Brassica rapa var. perviridis), radish (Raphanus sativus), Japanese horseradish (Wasabia japonica), etc.

Solanaceae: tobacco (Nicotiana tabacurn), eggplant (Solanum melongena), potato (Solanum tuberosum), tomato (Solanum lycopersicum), red pepper (Capsicum annuum), petunia (Petunia hybrida), etc.

Leguminosae: soybean (Glycine max), pea (Pisum sativum), broad bean (Vicio faba), Japanese wisteria (Wisteria floribunda), peanut (Arachis hypogaea), Bird's foot trefoil (Lotus corniculatus var. japonicus), kidney bean (Phaseolus vulgaris), adzuki bean (Vigna angularis), acacia (Acacia), etc.

Compositae: florists' daisy (Chrysanthemum morifolium), sunflower (Helianthus annuus), etc.

Arecaceae: oil palm (Elaeis guineensis and Elaeis oleifera), coconut (Cocos nucifera), date (Phoenix dactylifera), wax palm (Copernicia), etc.

Anacardiaceae: Japanese wax tree (Rhus succedanea), cashew (Anacardium occidentale), Japanese lacquer (Toxicodendron vernicifluum), mango (Mangifera indica), pistachio (Pislacia vera), etc.

Cucurbilaceae: pumpkin (Cucurbita maxima, Cucurbita moschata, and Cucurbita pepo), cucumber (Cucumis sativus), snake gourd (Trichosanthes cucumeroides), bottle gourd (Lagenaria siceraria var. gourda), etc.

Rosaceae: almond (Amygdalus communis), rose (Rosa), strawberry (Fragaria), cherry (Prunus), apple (Malus pumila var. domeslica), etc.

Caryophyllaceae: carnation (Dianthus caryophyllus), etc.

Salicaceae: poplar (Populus trichocarpa, Populus nigra, and Populus tremula), etc.

Poaceae: corn (Zea mays), rice (Oryza sativa), barley (Hordeum vulgare), wheat (Triticum aestivum), bamboo (Phyllostachys), sugarcane (Saccharum officinarum), napier grass (Pennisetum purpureum), erianthus (Erianthus ravennae), miscanthus (Japanese silver grass) (Miscanthus virgatum), sorghum (Sorghum), switchgrass (Panicum), etc.

Liliaceae: tulip (Tulipa), lily (Lilium), etc.

Among them, it is preferred to target an edible crop such as a plant of the family Poaceae or Leguminosae. Such an edible crop can be stably cultivated and harvested without being influenced by climate change, to thereby realize stable food supply. It is also preferred to target an energy crop, such as sugarcane, corn, rapeseed, or sunflower, which may be used as a raw material for biofuel. The cultivation range or cultivation conditions of an energy crop can be drastically expanded by improving the environmental stress tolerances of the energy crop. Specifically, it becomes possible to cultivate the energy crop even under a land or an environmental factor (e.g., average temperature and concentrations of salt contained in soil) that makes it impossible to cultivate the wild type. Thus, cost reduction of biofuel such as bioethanol, biodiesel, biomethanol, bio-DME, bio-GTL (BTL), and biobutanol can be achieved.

As described above, the plant disclosed in the present specification can also be obtained by breeding based on conventional mating, in addition to artificial gene engineering. The plant disclosed in the present specification has one or two or more improved environmental stress tolerances and has the tolerances to environmental stresses at a more practical level.

### (Method for Conferring Environmental Stress Tolerance to Plant and Method for Producing Plant)

The method for conferring environmental stress tolerances to a plant and the method for producing a plant provide with environmental stress tolerances, disclosed in the present specification, can comprise the step of increasing the expression of the first gene and/or the second gene which is an endogenous or foreign gene. This enhancement step can be carried out, as already mentioned, by combining breeding processes such as transformation and/or mating or mutation for the target plant. Particularly, for the production of a plant derived from mating, it is preferred to comprise the step of selecting the plant derived from mating by using the expression of the first gene and/or the second gene as an index. Efficient breeding and production are achieved by using a tolerance to stress as well as the expression patterns of these genes as indexes in various evaluations.

### (Method for Producing Crop)

The method for producing a crop disclosed in the present specification can comprise the step of cultivating a crop which is the plant disclosed in the present specification.

According to the present production method, various useful crops such as edible crops and energy crops can be stably cultivated and harvested, even under an environment where various environmental stresses including salt stress may occur, by circumventing or suppressing adverse effects caused by climate change.

Those skilled in the art can carry out the cultivation step in the present production method by appropriately selecting conditions, etc., according to the type of the crop.

### (Method for Screening Plant)

The method for screening plants disclosed in the present specification can comprise the steps of: screening one or two or more plants by using, as an index, the expression of one or two or more genes selected from a gene group consisting of the first gene subgroup and the second gene subgroup; and evaluating the salt tolerance of the plants having high expression levels of the one or two or more genes. According to this screening method, candidate plants that are likely to be excellent in salt tolerance can be primarily screened for easily by using the gene disclosed in the present specification such as the Os12g0150200 gene as an index. The candidate plants can be further evaluated for their salt tolerance to obtain a plant excellent in salt tolerance. Particularly, the gene including the Os12g0150200 gene tends to be also highly expressed under conditions that are not stress conditions such as high-salinity (as to NaCl) conditions (i.e., under non-stressed conditions). Thus, the candidate plants can also be screened for easily under non-stressed conditions.

In the screening step, the expression of the gene such as the Os12g0150200 gene is used as an index to select a plant having a high expression level of this gene as compared with the expression level in, for example, a general variety of the plant, typically, a wild type usually used as the plant, a typical cultivar, a model plant, or the like. The screening conditions may be high-salinity conditions generally used in salt tolerance evaluation or may be usual conditions as to salt concentrations. The gene including the Os12g0150200 gene tends to be also highly expressed, as mentioned above, under non-salt stress conditions. Therefore, the screening can also be performed under non-salt stress conditions.

The plants to be screened are not particularly limited and can be young plants or a portion thereof in consideration of screening efficiency. Examples thereof include a portion of young plants, such as leaves.

For the screening using the expression of the gene including the Os12g0150200 gene as an index, the level of mRNA or a protein which is an expression product of the gene can be used as the index. Methods for evaluating these are well-known to those skilled in the art. For the evaluation of the expression level of the gene, as described above, the expression level of the gene in a typical wild type of the plants to be screened, a variety thereof, or a variety to be modified can be used as a control. When the plants are, for example, rice. Nipponbare can be used. In the case of using, for example, Nipponbare as a control, a plant having approximately several to 100 times, preferably approximately 10 or more and 100 or less times the expression level of the control can be selected. Also, a plant having, for example, approximately 10 or more and 80 or less times the expression level can be selected. Furthermore, for example, the lower level of the expression level may be 10 or more times, may be 20 or more times, or may be 30 or more times. The upper limit of the expression level may be 70 or less times, may be 60 or less times, or may be 50 or less times.

A plant having a high expression level of the Os2g0150200 gene can be further evaluated for its salt tolerance under salt stress conditions. A plant reliably excellent in salt tolerance can be screened for by further conducting the evaluation of salt tolerance. For the evaluation of salt tolerance, a known salt tolerance evaluation approach as disclosed in Examples mentioned later can be appropriately selected and used according to a purpose, etc.

The plant excellent in salt tolerance that can be thus screened for is useful in itself. Specifically, the plant may have a variation or other modifications to enhance the expression of the gene such as the Os12g0150200 gene, in the coding region of the gene such as the Os12g0150200 gene and a region that is located upstream and/or downstream of the coding region and involved in expression control. Such a factor that contributes to enhancement in the expression level of the gene such as the Os12g0150200 gene, for example, a variation or the like on the chromosomes, is identified and thereby useful in the development of a plant excellent in salt tolerance, etc. In short, a DNA region containing such a factor can be used for improvement in salt tolerance in other varieties or plants. A known plant breeding method such as mating or transformation can be used in the application of such a DNA region to other plants or varieties.

As described above, the first gene and/or the second gene disclosed in the present specification can provide practical environmental stress tolerances to a plant.

### EXAMPLES

Hereinafter, the present invention will be described in more detail with reference to Examples. However, the technical scope of the present invention is not intended to be limited by these Examples.

### Example 1

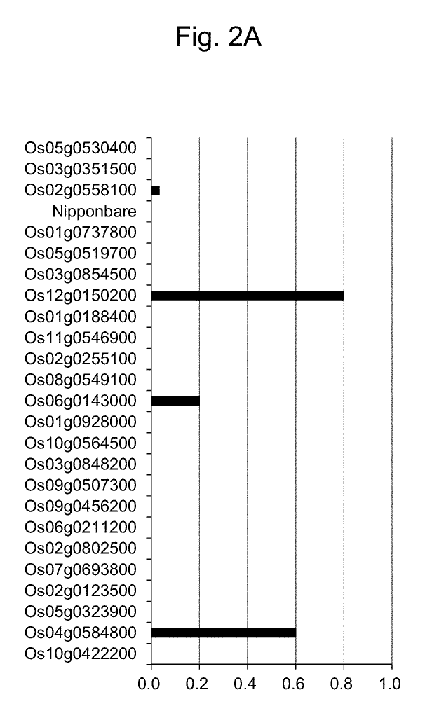

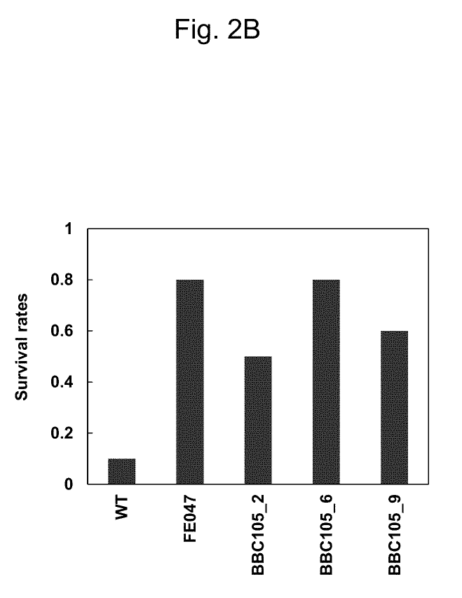

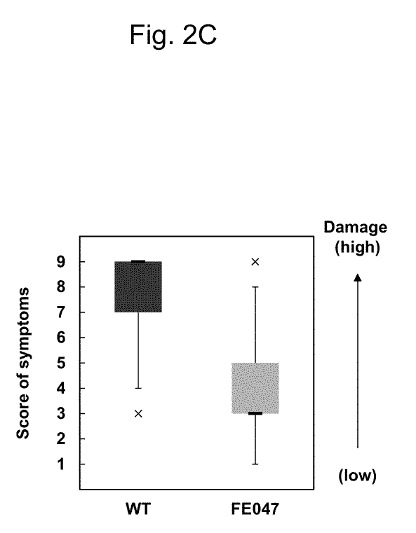

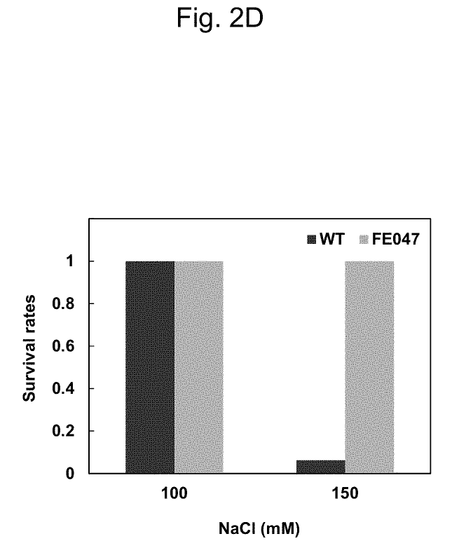

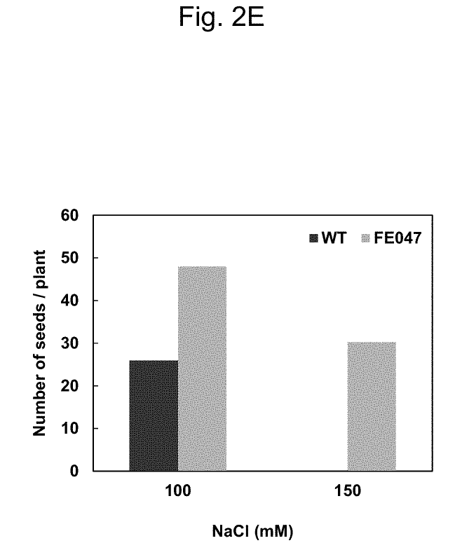

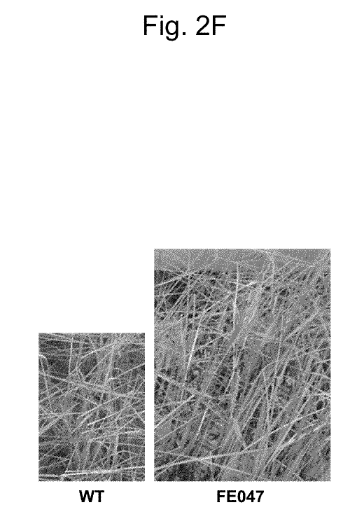

Rice full-length cDNA-overexpressing (FOX) rice lines (Nakamura. et al., 2007; Hakata, et al. 2010; and Tsuchida-Mayama, et al., 2010) distributed by National Institute of Agrobiological Sciences were used to select a salt-tolerant line. Chaff was removed from the seeds of wild-type rice (variety: Nipponbare; Oryza sativa cv. Nipponbare) or each selected FOX rice line, and the resulting seeds were surface-sterilized, then put on a Murashige & Skoog (MS) basal solid medium [containing 1% sucrose, 0.25% gellan gum, and 0.05% MES-KOH (pH 5.8)] prepared in a mayonnaise bottle of 13 cm in height, and aseptically germinated and grown. Seedlings were cultivated at 25° C. under 14-hour light (white light: 4000 lux) and 10-hour dark conditions. The bases (4 cm long) of the aerial parts of 1-week-old seedlings were aseptically cut out and transplanted to a fresh MS basal solid medium. A 600 mM aqueous NaCl solution (final concentration: 300 mM) was aseptically added thereto in an amount equal to the amount of the medium. After growing for 5 weeks under this salt stress condition, the plants were dipped in tap water and acclimatized for 1 week, and examined for their survival rates. The survival rate of each overexpressing line is shown in FIG. 2A. Nipponbare is the wild type.

As shown in FIG. 2A, two lines among a large number of FOX rice lines exhibited a good score. Full-length cDNA encoded by the genes Os12g0150200 and Os04g0584800 introduced in these two lines was identified by the method of the literatures (Nakamura, et al., 2007; Hakata, et al., 2010; and Tsuchida-Mayama, et al., 2010).

The full-length cDNA (full-length cDNA encoded by Os12g0150200: accession number AK064287) introduced in the FOX rice line that exhibited salt tolerance was amplified by PCR using the primers shown in Table 1, subcloned into a pENTR/D-TOPO vector (Thermo Fisher Scientific Inc.), and then inserted to downstream of the rice actin promoter of a binary vector pSMAHdN637L-GateA. The obtained construct was used in the Agrobacterium-mediated transformation of rice (Nipponbare) to obtain novel Os12g0150200 cDNA-overexpressing lines (BBC105 line group).

The progeny seeds of the wild type and the Os12g0150200 cDNA-overexpressing lines (FE047 line and BBC105 line group) were used to conduct the same salt tolerance test as mentioned above. After growing for 5 weeks under the salt stress condition, the plants were dipped in tap water and acclimatized for 1 week, and examined for their survival rates. The obtained results of testing the wild type. FE047, BBC105_2, BBC105_6, and BBC105_9 are shown in FIG. 2B. The number of individuals tested (n) are 10 for WT and FE047, 4 for BBC105_2, and 5 for BBC105_6 and BBC105_9.

As shown in FIG. 2B, all of the Os12g0150200 cDNA-overexpressing lines FE047, BBC105_2, BBC105_6, and BBC105_9 exhibited a high survival rate with respect to the wild type.

Next, the wild-type rice and the FE047 line were each cultivated under salt treatment in a closed greenhouse, and the salt tolerance was compared therebetween.

The salt tolerance test and salt tolerance evaluation basically followed the method of Thomson et al. (2010). Husked rice seeds treated with Benlate were placed on wet filter paper and germinated by incubation at 30° C. for 2 days in the dark. Seedlings were transferred onto floats in a greenhouse and grown for 3 days in deionized water. Then, 240 seedlings per float were grown in 10 L of a Yoshida medium. The electrical conductivity (EC) of the hydroponic solution was set to 6 dSm⁻¹ for the first 3 days and then set to 12 dSm⁻¹ by the addition of sodium chloride. The EC and pH were adjusted by the addition of water and NaOH every 2 to 3 days. After 2 weeks, damage caused by salt stress was evaluated according to the salinity evaluation score of Thomson et al. (2010). The obtained scores are shown in FIG. 2C. The box plots represent an interquartile range (IQR). Values that did not fall within the range of 1.5×IQR are shown as outliers outside the first quartile and the third quartile. Whiskers represent the range from the smallest value to the largest value. Median values are indicated by thick horizontal bars, n=38 (WT) and n=20 (FE047).

As shown in FIG. 2C, the damage caused by the salt treatment was suppressed in the selected FE047 line as compared with the wild type.

Further, the wild-type rice and the FE047 line were each cultivated in salt-damaged soil, and the salt tolerance was compared therebetween. The 4-week-old plants were transplanted to Wagner pots of 23 cm in diameter containing 10 L of soil and cultivated. The number of seedlings transplanted was up to 8 individuals per pot. The pots were arranged in a tank and filled with water, and the plants were grown for 2 weeks. Then, the water was replaced with 100 mM NaCl or 150 mM NaCl. EC and pH were adjusted by the addition of water and NaOH, respectively, every 2 to 3 days. The survival rates of rice in the experiment using salt-damaged soil containing 100 mM NaCl or 150 mM NaCl are shown in FIG. 2D, and the number of seeds per individual is shown in FIG. 2E, n=17 (WT), n=4 (FE047, 100 mM), and n=3 (FE047, 150 mM). The photographs of the plants of the wild-type rice and the FE047 line taken 40 days after the addition of 150 mM NaCl are shown in FIG. 2F.

As shown in FIGS. 2D to 2F, the FE047 line exhibited a favorable survival rate, fertility, and luxuriant development as compared with the wild type. These results demonstrated that the Os12g0150200 cDNA-overexpressing line has a very practical tolerance to salt stress.

### Example 2

In this Example, the accumulation and induction of jasmonic acid (JA), active jasmonic acid (JA-Ile), and inactive jasmonic acid (120H-JA-Ile) after wounding of the wild type and the FE047 line were evaluated.

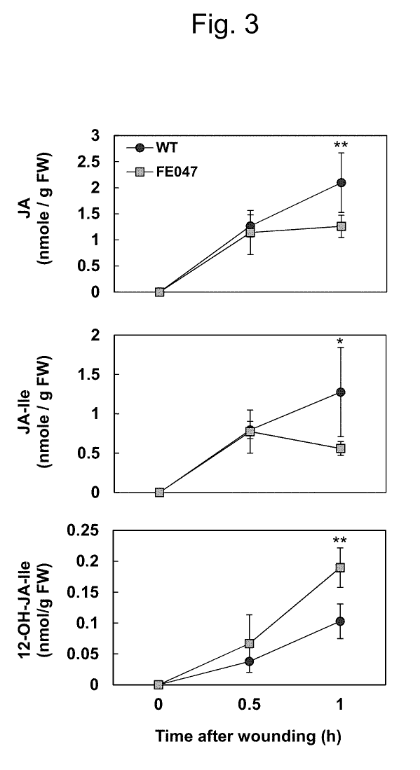

A half lamina of the third leaf of each rice seedling was cut out and immediately frozen in liquid nitrogen. The remaining half lamina of the third leaf was wounded by pinching with tweezers 20 times. Then, the whole seedling was incubated at room temperature in a humid chamber, and the wounded leaf was recovered and frozen in liquid nitrogen. Mini-Bead Beater-8 (Bio Spec Products Inc.) was used, and the frozen leaf tissue was disrupted in a microcentrifuge containing stainless beads. The extraction of JA and its derivatives from the tissue with 99.5% ethanol was performed overnight at 4° C. in the dark. The residue was removed by centrifugation (20,000×g) at room temperature for 5 minutes, and the ethanol solution as a supernatant was preserved at 4° C. Before measurement, the ethanol solution was dried using nitrogen gas, and the sample was then dissolved in water. The residue was further removed by centrifugation (15,000×g) at room temperature for 20 minutes, and the supernatant fraction was used in measurement by UPLC/TOFMS. The amounts of JA, JA-Ile, and 120H-JA-Ile per body weight of the leaf tissue before the wounding and after the wounding (mean±standard deviation; n≧6) are shown in FIG. 3. In this figure, analytes exhibiting the significant difference between the wild type and the FE047 line are indicated by asterisks (**: p-value<0.01; *: p-value<0.05, Student's t-test).

As shown in FIG. 3, in the wild type, JA and its active form were increased with time by the wounding while its inactive form was also gradually increased. By contrast, in the FE047 line, JA and its active form were initially increased, but then decreased. Along with this decrease, a tendency in which the inactive form was increased was observed.

These results suggested that the protein encoded by Os12g0150200 has the activity of converting JA from an activity form to an inactive form.

### Example 3

In this Example, the responses of the wild type and the FE047 line to JA and coronatine (COR) were evaluated. COR is known to activate JA response by binding to COI1 receptor, as with active jasmonic acid.

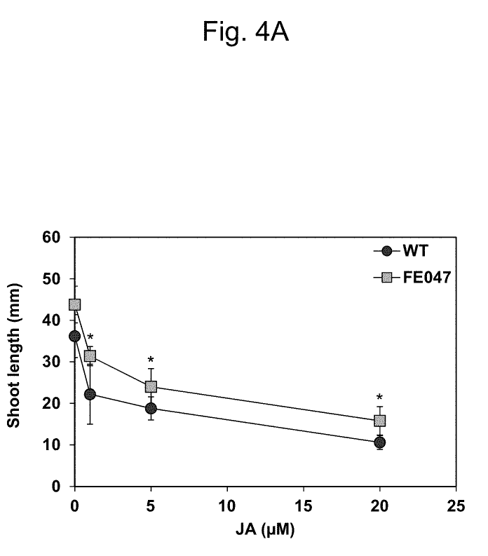

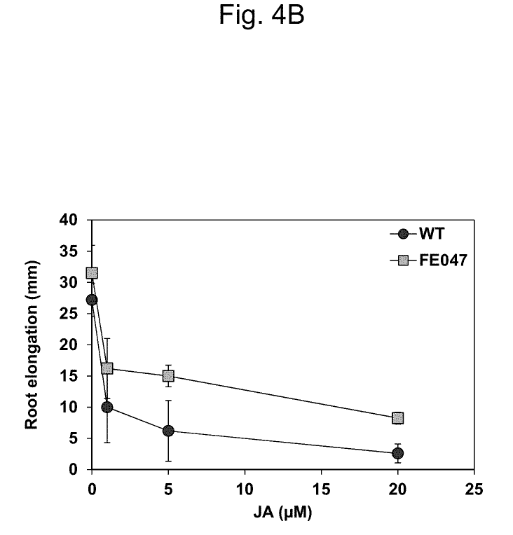

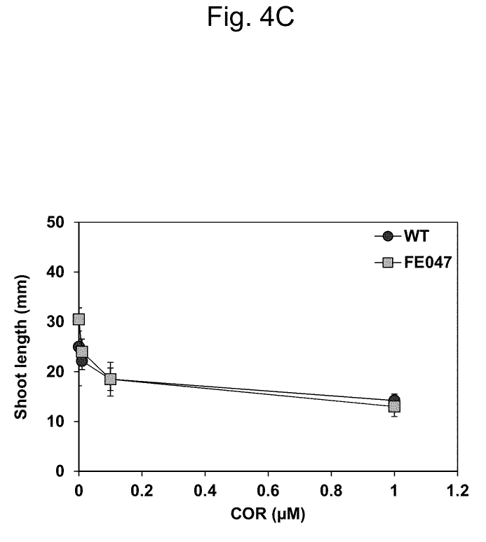

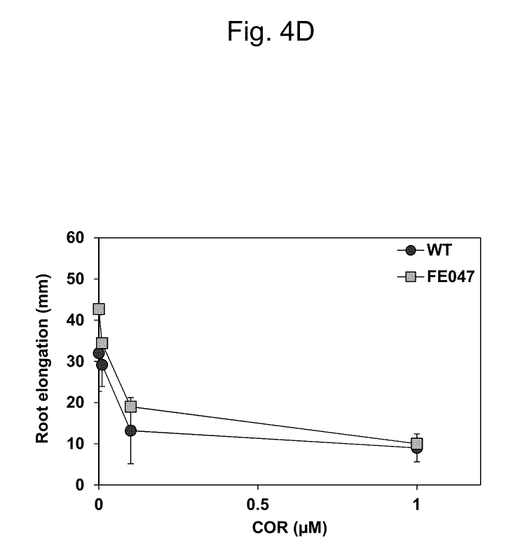

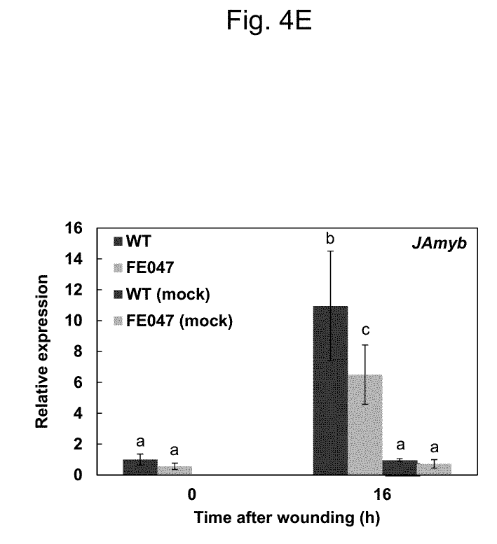

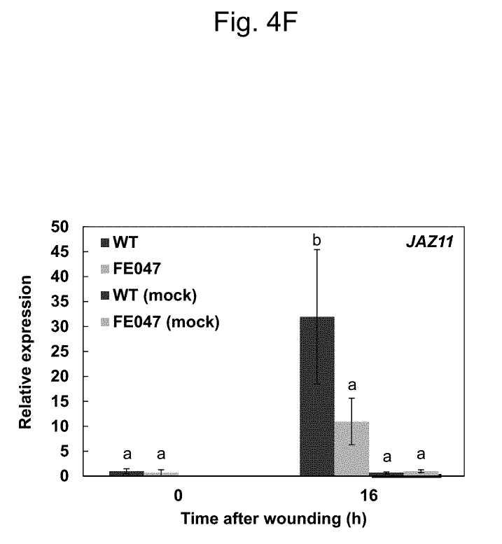

Chaff was removed from the seeds of the wild type or the FE047 line, and the resulting seeds were surface-sterilized, then inoculated to MS basal solid media containing varying concentrations of JA or COR prepared in a mayonnaise bottle of 13 cm in height, and aseptically grown. The shoot lengths of 3-day-old seedlings grown in the presence of JA are shown in FIG. 4A, and the shoot lengths of 3-day-old seedlings grown in the presence of COR are shown in FIG. 4C. Root elongation from 2 days to 3 days after the inoculation in the presence of JA is shown in FIG. 4B, and root elongation from 3 days to 4 days after the inoculation in the presence of COR is shown in FIG. 4D. Mean±standard deviation (n>4) is shown for each. Analytes exhibiting the significant difference between the wild type and the FE047 line are indicated by asterisks (**: p-value<0.01; *: p-value<0.05, Student's t-test).

As shown in FIGS. 4A and 4B, the FE047 line had a smaller degree of suppression of shoot length or root elongation than that of the wild type in the presence of JA, and weakened JA response was observed. By contrast, as shown in FIGS. 4C and 4D, no difference was detected in the degrees of shoot length and root elongation between the wild type and the FE047 line in the presence of COR.

Also, the responses of the wild type and the FE047 line to damage were evaluated by using JAmyb and JAZ11 exhibiting would-inducible expression as gene markers. The leaves of 7-day-old seedlings were mechanically wounded by pinching with tweezers 20 times and incubated for 16 hours in a humid chamber. Then, the leaf tissues were frozen in liquid nitrogen and preserved at −80° C. The extraction and purification of total RNA were performed using RNeasy Plant Mini kit (Qiagen N.V.). Reverse transcription from the RNA was performed using QuantiTect Rev Transcription kit (Qiagen N.V.). The obtained single-stranded cDNA was amplified by PCR using PrimeStar HS DNA polymerase (Takara Bio Inc.) and GeneAmp PCR System 9700 (Applied Biosystems, Inc.) or real-time PCR using Power SYBR Green PCR Master Mix (Applied Biosystems, Inc.) and Mx3000P (Agilent Technologies, Inc.). The primers used are shown in the table below. The expression levels of JAmyb mRNA and JAZ11 mRNA were normalized by the expression level of UBC mRNA, and the expression levels in the wounded leaves were determined as relative values to the levels in untreated leaves. The results are shown in FIGS. 4E and 4F. The results are indicated by mean±standard deviation (n=5). Also, a statistical significant difference test was conducted by multiple comparisons based on the Tukey-Kramer test. In the figures, a to c represent the significant difference between the groups (p-value<0.01).

TABLE 1
For generation of transgenic rice plants

| Name | Sequence | Usage |
|---|---|---|
| pAct-1F | AATCCCTCAGCAT | Genotyping |
| | TGTTCATC | |
| Tnos-1R | CCCATCTCATAAA | Genotyping to |
| | TAACGTCATGC | amplify cDNA |
| | | from NIAS clone |
| CACC-SP6 | CACCATTTAGGTG | (002-105-H01) |
| | ACACTATAGAA | to amplify cDNA |
| | | from NIAS clone |
| T7 | TAATACGACTCAC | (002-105-H01) |
| | TATAGGG | |

For expression analysis

| | Sequence | | |
|---|---|---|---|
| Name | (5′ to 3′) | Usage | Gene or Locus |
| AK064287 | GGGCTCCCAGTGA | Cyp94C2b | Os12g0150200 |
| (1618F) | AGATCAG | | |
| AK064287 | TCCAATTTGTTGA | | |
| (1705R) | TACAGTGAATGAA | | |
| JAmyb | CTGGTGTAACAAT | JAMyb | Os01g0853400 |
| (F674) | GGCCACTG | | |
| Jamyb | TCAAATCTTGTGA | | |
| (R775) | CTCCGACG | | |
| OsJAZ11_F2 | TGAGGAAGACCAT | Os-JAZ11 | Os03g0180900 |
| | GGAGCTCTT | | |
| OsJAZ11_R2 | TCTCGGTGATCTC | | |
| | CTTCCTTTC | | |
| UBC-F | CCGTTTGTAGAGC | UBC | Os02g0634800 |
| | CATAATTGCA | | |
| UBC-R | AGGTTGCCTGAGT | | |
| | CACAGTTAAGTG | | |
| RT- | AAGGCCGAAGAGG | 25S | AK119809 |
| 25SrRNAF | AGAAAGGT | ribosomal | |
| RT- | CGTCCCTTAGGAT | RNA | |
| 25SrRNAR | CGGCTTAC | | |
| SGR-F | CTACCAAACCGAG | SGR | Os09g0532000 |
| | CCAAAAT | | |
| SGR-R | ACCAAAACGACTC | | |
| | TTGACAGC | | |
| RR10-F | CAATAGATGCCTG | OsRR10 | Os12g0139400 |
| | GAGGAAGGA | | |
| RR10-R | TCATGTCGGAGAG | | |
| | CCGAACT | | |

As shown in FIGS. 4E and 4F, the expression of both the JA-responsive genes JAmyb and JAZ11 was increased by the wounding in the wild type, but was strongly suppressed in the FE047 line.

These results demonstrated that the protein encoded by Os12g0150200 converts active JA to an inactive form in response to JA, but does not inactivate coronatine. These results and the molecular phylogenetic tree and the alignment based on the amino acid sequence of the protein encoded by Os12g0150200 shown in FIG. 1 suggested that Os12g0150200 encodes a protein having the activity of converting active jasmonic acid to an inactive form.

### Example 4

In this Example, the leaf etiolation of the wild type and the FE047 line under a salt stress environment was evaluated.

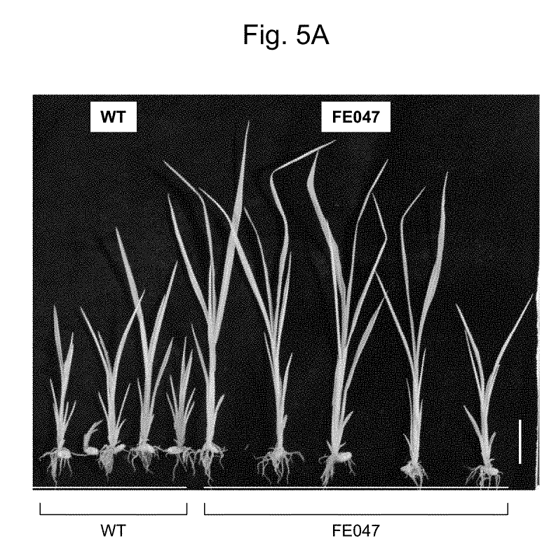

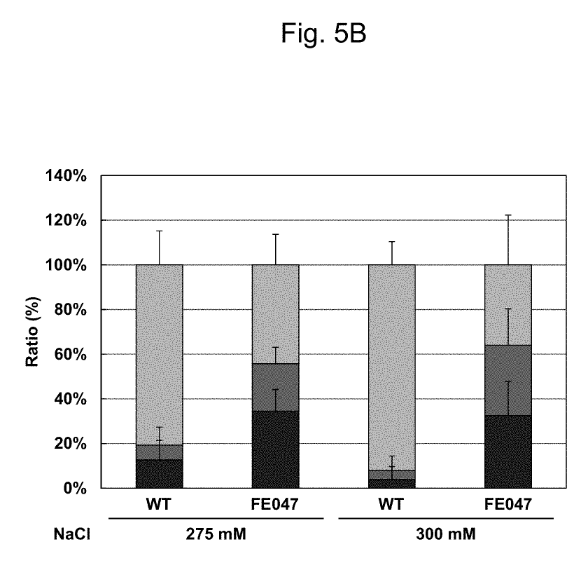

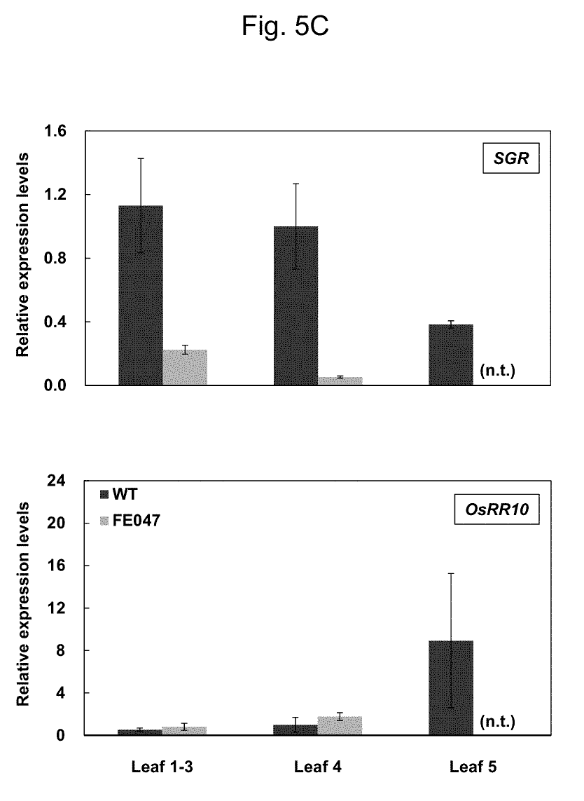

Chaff was removed from rice seeds, and the resulting seeds were surface-sterilized, then inoculated to a MS basal solid medium containing 300 mM NaCl prepared in a mayonnaise bottle of 13 cm in height, and grown for 33 days. The resulting plants are shown in FIG. 5A. The left 5 individuals are of the wild type, and the right 5 individuals are of the FE047 line. The bar represents 2 cm.

A similar experiment was conducted using a MS basal solid medium containing 275 mM NaCl. The degree of etiolation of leaves in the case of using each salt concentration was indicated by the ratios of the colors of the leaves. The ratio of yellow or brown leaves (yellow box), the ratio of yellow-green leaves (light green box), and the ratio of green leaves (dark green box) are shown in FIG. 5B. The results were indicated by mean±standard deviation (n==10).

Similarly, the wild type and the FE047 line were each cultivated for 33 days using a MS basal solid medium containing 250 mM NaCl. The expression of an aging marker gene STAYGREEN (SGR) in etiolated leaves (mixture of the first to third leaves) and young leaves (the fourth and fifth leaves) was examined. The expression level of SGR was examined by qRT-PCR, and this was normalized by the expression level of UBC and indicated by a relative value to the expression level in the fourth leaf of the wild type. The SGR gene encodes a rate-limiting enzyme that degrades chlorophyll. The expression analysis was conducted in the same way as in Example 3.

Moreover, the expression pattern of a cytokinin-responsive marker gene OsRR10 was similarly indicated. The cytokinin is a hormone that suppresses the aging of leaves.

The results about the SGR marker are shown in FIG. 5C (upper diagram), and the results about the OsRR10 marker are shown in FIG. 5C (lower diagram). The results were indicated by mean±standard deviation. The leaves were numbered in order from oldest to youngest.

As shown in FIGS. 5A and 5B, yellow leaves under salt stress were fewer in number in the Fe047 line than in the wild type, and the suppression of growth was also lower. As shown in FIG. 5C, the expression of the aging marker was largely suppressed in the FE047 line as compared with the wild type, whereas the activation of the cytokinin-responsive marker serving as an index for the suppression of leaf aging was not observed.

These results demonstrated that in the FE047 line, etiolation is suppressed by activating the functions of cytokinin.

### Example 5

In this Example, the wild type, the FE047 line (T2 generation), and the independent Os12g0150200 (CYP94C2b) cDNA-overexpressing lines (BBC105 line group; T1) prepared in Example 1 were used to evaluate CYP94C2b expression levels and salt tolerance levels.

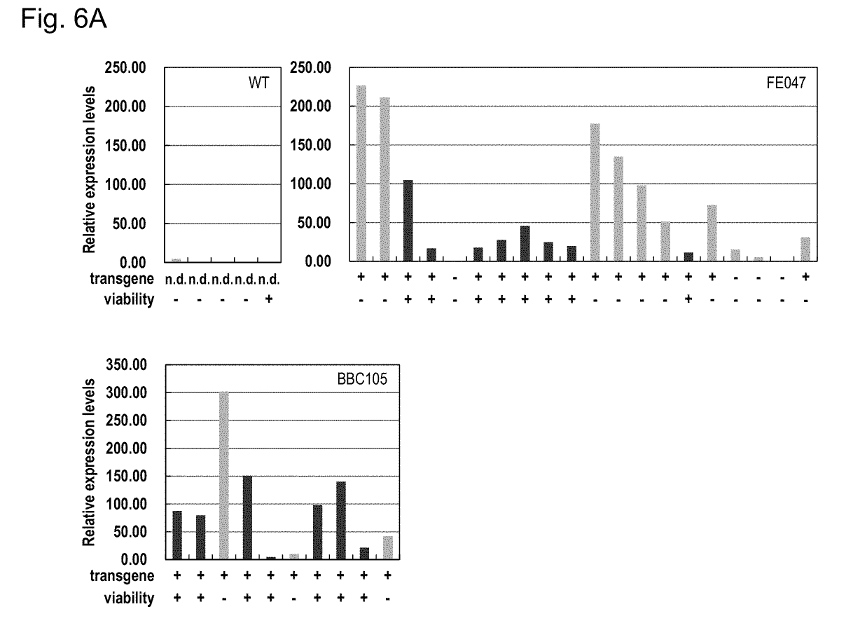

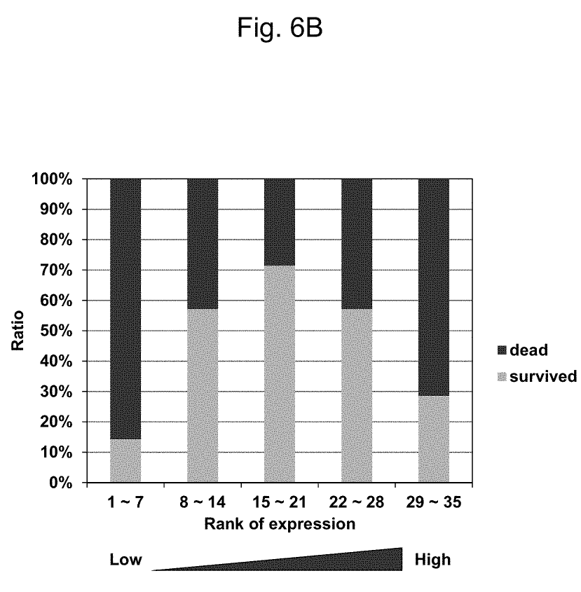

RNA was extracted from the leaves of 7-day-old seedlings cultivated under non-salt stress conditions. The expression level of CYP94C2b was determined by qRT-PCR using the primers shown in Table 1. The CYP94C2b expression level was normalized by the expression level of 25S rRNA, indicated by a relative value to the mean of 5 wild-type individuals, and shown in FIG. 6A. The presence or absence of the transgene (Os12g0150200) in the tested plants, and viability in the presence of the same salt stress as in Example 1 are indicated by “+/−” below the graph. The presence or absence of the transgene was examined by PCR using the primers (for genotyping) shown in Table 1 and genomic DNA as a template. n.d. denotes not detected.

In addition, the 35 rice individuals in total of FIG. 6A were ranked by using the CYP94C2b expression level as an index. A survival rate in the presence of salt stress at each rank (involving 7 individuals) when the expression level was divided into 5 categories is shown in FIG. 6B. Ranks 1 to 7 showed individuals exhibiting a lowest expression level, which included the wild type (5 individuals) and individuals having no transgene (2 individuals). By contrast, individuals of ranks 29 to 35 exhibited a highest expression level and exhibited approximately 150 or more times the expression in the wild type.

These results demonstrated that a higher expression level of CYP94C2b than that in the wild type elevates environmental stress tolerances to salt stress and the like and it is desirable that the degree of enhancement in the expression level should be a degree that does not exceed 150 times that in the wild type. The expression level is preferably 10 or more times and 150 or less times, more preferably 10 or more times and 100 or less times the expression level in the wild type.

### Example 6

The cDNA of the transgene Os12g0150200 or Os04g0584800 in each selected salt-tolerant line was inserted to an overexpression vector and then introduced in Nipponbare to newly produce transformed lines according to Example 1. Next-generation seeds were used to conduct a salt tolerance test. Five individuals each of independent 4 lines per gene were subjected to the test.

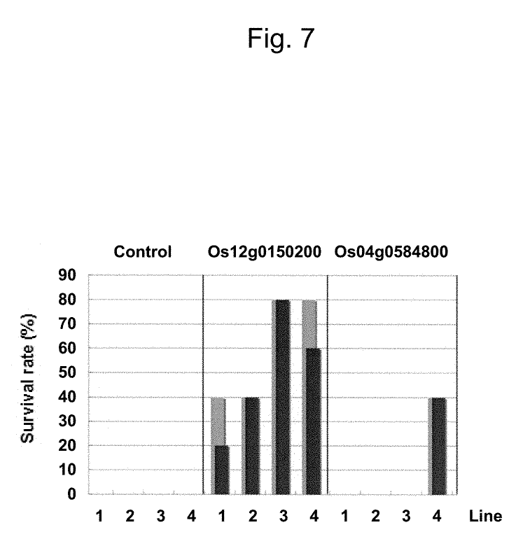

The seeds of the wild type (Nipponbare) or the transformed rice were surface-sterilized, then put on a MS basal solid medium [containing 1% sucrose, 0.25% gellan gum, and 0.05% MES-KOH (pH 5.8)] prepared in a mayonnaise bottle of 13 cm in height, and aseptically germinated and grown. Seedlings were cultivated at 25° C. under 14-hour light (white light: 4000 lux) and 10-hour dark conditions. The bases (4 cm long) of the aerial parts of 1-week-old seedlings were cut out and transplanted to a fresh MS basal solid medium. Further, a 600 mM aqueous NaCl solution (final concentration: 300 mM) was aseptically added thereto in an amount equal to the amount of the medium. At the point when the plants were grown for 5 weeks under this salt stress condition, their survival rates were evaluated. Then, the plants were dipped in tap water and acclimatized for 1 week, and further evaluated for their survival rates. The results are shown in FIG. 7. The survival rates under salt stress are indicated by gray bars, and the subsequent survival rates in the absence of salt stress are indicated by black bars.

As shown in FIG. 7, all of the lines having Os12g0150200 introduced therein were able to be confirmed to have salt stress tolerance, and some of the lines having Os04g0584800 introduced therein were also confirmed to exhibit salt stress tolerance.

### Example 7

In this Example, the Os12g0150200-overexpressing line (FE047) and the Os04g0584800-overexpressing line (CU099) prepared in Example 1 were evaluated for their environmental stress tolerances under other conditions.

### (1) Salt Stress Tolerance Test (Closed Greenhouse)

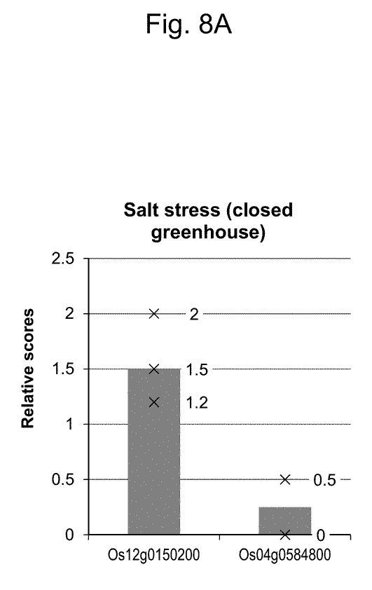

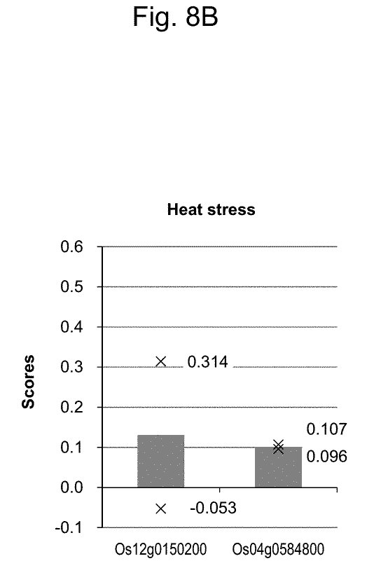

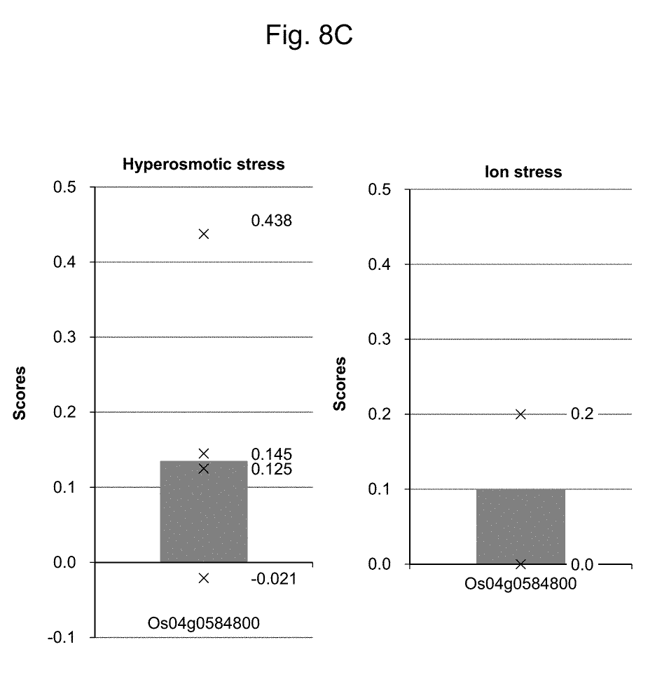

A hydroponic cultivation test similar to the salt tolerance test of International Rice Research Institute (IRRT) was conducted (Thomson et al., 2010). On 3 days and 6 days after germination, NaCl was administered in stages, and damage on plants was then scored. In the salt tolerance test in a closed greenhouse, Nipponbare and a salt-tolerant line Kyudai-asahi 3 were used as comparative controls, and the plants were evaluated using the evaluation score described in the literature. Further, the degree of salt stress tolerance of each overexpressing line was calculated according to the expression (Evaluation score of Nipponbare—Evaluation score of each overexpressing line)/(Evaluation score of Nipponbare—Evaluation score of Kyudai-asahi 3). Specifically, the degree of salt stress tolerance was indicated by a relative value when the degree of the tolerance of Nipponbare was defined as “0” and the degree of the tolerance of Kyudai-asahi 3 was defined as “I”. The results are shown in FIG. 8A. In the graph, the plot shows a value obtained by repetitive tests, and the box represents a median value (the same holds true for FIGS. 8B and 8C).

As shown in FIG. 8A, the Os12g0150200-overexpressing line also exhibited a high degree of salt stress tolerance in the salt tolerance test in a closed greenhouse, and the Os04g0584800-overexpressing line also exhibited an excellent degree of salt stress tolerance, albeit lower than that of the Os12g0150200-overexpressing line.

### (2) Heat Stress Tolerance Test

Rice seeds were hydroponically cultivated at 28° C. both for 3 days in ultrapure water and for 17 days in a Yoshida medium. The obtained plants were cultivated at 42° C. for 7 days, brought back to 28° C., and cultivated at this temperature for 7 days. Their survival rates were compared with the survival rate of the wild type. The degree of heat stress tolerance provided by the overexpression of each gene was indicated by a value determined by subtracting the survival rate (0.44 to 0.75) of the wild type (Nipponbare) from the survival rate of each overexpressing line. The results are shown in FIG. 83.

As shown in FIG. 8B, both of the Os12g0150200-overexpressing line and the Os04g0584800-overexpressing line were found to have heat stress tolerance better than that of the wild type.

### (3) Hyperosmotic Stress Tolerance Test

Rice seeds of the Os04g0584800-overexpressing line (CU099) or the wild type were hydroponically cultivated for 3 days in ultrapure water and further for 15 days in a Yoshida medium. The obtained plants were cultivated for 7 days in a medium containing 26% polyethylene glycol (PEG: average molecular weight of 4,000) and further for 4 days in a medium free from PEG. Their survival rates were compared. The degree of hyperosmotic stress tolerance provided by the overexpression of the Os04g0584800 gene was indicated by a value determined by subtracting the survival rate (0.21 to 0.69) of the wild type (Nipponbare) from the survival rate of the Os04g0584800-overexpressing line (CU099). The results are shown in FIG. 8C.

### (4) Ion Stress Tolerance Test

Sterilized seeds of the Os04g0584800-overexpressing line (CU099) or the wild-type rice were inoculated to a MS medium and grown for 7 days. The aerial parts (4 cm) of the obtained plants were cut out, transplanted to a MS medium containing 40 mM LiCl, and grown for 2 weeks. Their survival rates were compared. The degree of ion stress tolerance provided by the overexpression of the Os04g0584800 gene was indicated by a value determined by subtracting the survival rate (0 for all samples) of the wild type (Nipponbare) from the survival rate of the Os04g0584800-overexpressing line (CU099). The results are also shown in FIG. 8C.

As shown in FIG. 8C, the Os04g0584800-overexpressing line (CU099) was found to have excellent tolerances to hyperosmotic stress and ion stress.

These results demonstrated that the Os12g0150200-overexpressing line has excellent salt stress tolerance and heat stress tolerance and has excellent tolerances to a plurality of stresses including salt stress. The salt stress tolerance and the heat stress tolerance are also stress tolerances often required at the same time for plants under an outdoor environment. Thus, the increased expression of the Os12g0150200 gene or a gene equivalent to the gene can provide very practical stress tolerances to a plant.

Furthermore, the Os04g0584800 gene was found to be able to exhibit tolerances to salt stress as well as heat stress, and further hyperosmotic stress and ion stress. Thus, the increased expression of the Os04g0584800 gene and a gene equivalent to the gene can provide very practical stress tolerances to a plant.

### Example 8

In this Example, the standard rice variety (Nipponbare) and salt-tolerant rice varieties (Heitai and Pokkali) were each subjected to the salt tolerance test (in a laboratory and in a greenhouse) according to Example 1, while the expression level of the Os12g0150200 (Cyp94C2b) gene in the leaf tissues of their seedlings was evaluated.

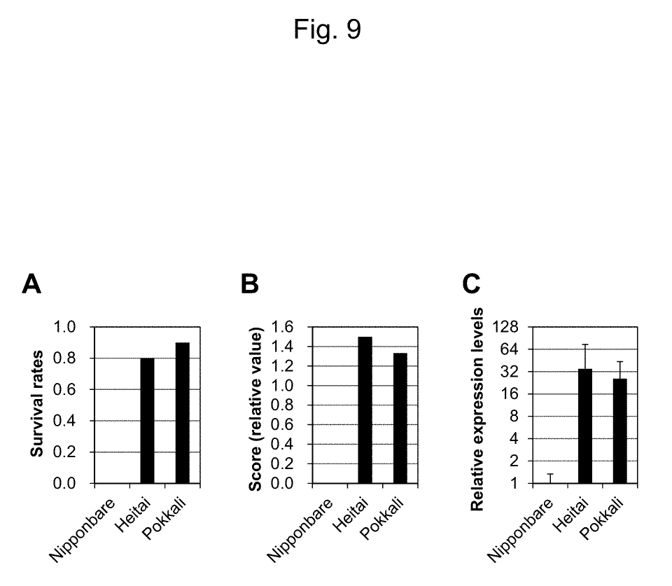

Specifically, for the salt tolerance test (in a laboratory), the bases of the aerial parts of 1-week-old seedlings of rice of each variety were transplanted to a MS basal solid medium. Then, a 600 mM aqueous NaCl solution (final concentration: 300 mM) was added thereto in an amount equal to the amount of the medium. After growing for 5 weeks, the plants were dipped in tap water and acclimatized for 1 week. Then, their survival rates were evaluated. The results are shown in A of FIG. 9. The number of individuals tested (n) was 10 for Nipponbare, 10 for Heitai, and 10 for Pokkali.

For the salt tolerance test (in a greenhouse), 20 individuals of rice of each variety were evaluated according to the salinity evaluation score of Thomson et al. (2010). Then, relative values were calculated using their respective median values and shown in a graph. The results are shown in B of FIG. 9. For the relative value calculation, the median values of the evaluation scores of varieties tested as comparative references (Nipponbare and Shinriki 7) and the following expression were used.

Relative value=(Median value of Nipponbare−Median value of the sample)/(Median value of Nipponbare−Median value of Shinriki 7)

In addition, leaf tissues (under non-stressed conditions) of 1-week-old seedlings of rice of each variety were frozen in liquid nitrogen and preserved at −80° C. The extraction and purification of total RNA therefrom were performed using RNeasy Plant Mini Kit (Qiagen N.V.). Then, reverse transcription from the RNA using QuantiTect Rev Transcription Kit (Qiagen N.V.) was performed. The obtained single-stranded cDNA was amplified by real-time PCR using SsoAdvanced Universal SYBR Green Supermix (Bio-Rad Laboratories, Inc.). The primers used are shown in Table 1. The expression level of Cyp94C2b mRNA was normalized by the expression level of 25S ribosomal RNA and indicated by a relative value to the expression level in Nipponbare. The results are shown in C of FIG. 9. The graph was indicated by mean±standard deviation (n=3).

As shown in A and B of FIG. 9, both of the existing salt-tolerant rice varieties Heitai and Pokkali exhibited a high survival rate with respect to Nipponbare in the salt tolerance test under the two conditions (in a laboratory and in a greenhouse). As shown in C of FIG. 9, Heitai and Pokkali were able to be confirmed to have a high expression level of the Os12g015(0200 (CYP94C2b) gene in the leaves of the plants grown under non-stressed conditions, as compared with the standard variety (Nipponbare). Heitai had 5 to 80 times the expression level in Nipponbare, and Pokkali had 10 to 45 times the expression level in Nipponbare. On the other hand, as shown in FIG. 6A, the Os12g0150200 (Cyp94C2b) gene-overexpressing plant having improved salt tolerance attained approximately 5 to 150 times the expression level of CYP94C2b in the wild type (Nipponbare).

These results demonstrated that the high expression of CYP94C2b can contribute to the high salt tolerance of the existing salt-tolerant variety Heitai or Pokkali. Furthermore, the high expression of CYP94C2b observed in the existing salt-tolerant rice varieties strongly supports feasible improvement in salt tolerance by the increased expression of CYP94C2b. For example, it is also considered possible to improve salt tolerance by screening for a variety having the high expression of the CYP94C2b gene and introducing a gene containing the coding region thereof and further, the expression control region in a cultivar.

In the present specification, papers given below are further referred to. The literatures already cited herein as well as these literatures are incorporated herein by reference in their entirety.
(1) Nakamura, H., Hakata, M., Amano, K., Miyao, A., Toki, N., Kajikawa, M. et al. (2007) A genome-wide gain-of function analysis of rice genes using the FOX-hunting system. Plant Mol. Biol. 65: 357-371.
(2) Hakata, M., Nakamura, H., Iida-Okada, K., Miyao, A., Kajikawa, M., Imai-Toki. N. et at. (2010) Production and characterization of a large population of cDNA-overexpressing transgenic rice plants using Gateway-based full-length cDNA expression libraries. Breed. Sci. 60: 575-585.
(3) Tsuchida-Mayama, T., Nakamura, H., Hakata, M. and Ichikawa, H. (2010) Rice transgenic resources with gain-of-function phenotypes. Breeding Sci. 60: 493-501.
(4) Thomson, M. J., de Ocampo, M., Egdane, J., Rahman M. A., Sajise. A. G., Adorada, D. L. et al. (2010) Characterizing the Saltol Quantitative Trait Locus for Salinity Tolerance in Rice. Rice 3: 148-160.

All publications, patents, and patent applications cited herein are incorporated herein by reference in their entirety.

We claim:

1. A plant having the increased expression of one or more genes selected from
a first gene subgroup consisting of Os12g0150200 gene, Os01g0858350 gene, Os05g0445100 gene, Os11g0151400 gene, AT3G48520 gene, and AT2G27690 gene, and functionally equivalent genes thereof, or
a second gene subgroup consisting of Os04g0584800 gene and functionally equivalent gene thereof.

2. The plant according to claim 1, wherein the plant has an enhanced environmental stress tolerance.

3. The plant according to claim 2, wherein the plant has enhanced salt stress tolerance.

4. The plant according to claim 3, wherein the plant further has an enhanced additional environmental stress tolerance.

5. The plant according to claim 1, wherein the plant has the increased expression of the first gene.

6. The plant according to claim 5, wherein the first gene is Os12g0150200 gene represented by SEQ ID NO: 1 or a functionally equivalent gene.

7. The plant according to claim 1, wherein the plant has the increased expression of the second gene.

8. The plant according to claim 7, wherein the plant has the increased expression of Os04g0584800 gene represented by SEQ ID NO: 25 or a functionally equivalent gene.

9. A plant having a nucleic acid encoding a protein introduced therein or having the increased expression of the nucleic acid, wherein
the protein is selected from the group consisting of:
(a) a protein comprising an amino acid sequence represented by any of SEQ ID NOs: 2, 4, 6, 8, 10, and 12;
(b) a protein comprising an amino acid sequence derived from the amino acid sequence represented by any of SEQ ID NOs: 2, 4, 6, 8, 10, and 12 by the deletion, substitution, addition, or insertion of one or more amino acids, and having (i) the activity of converting active jasmonic acid to inactive jasmonic acid or (ii) the activity of enhancing the environmental stress tolerances of plants;
(c) a protein comprising an amino acid sequence having 90% or higher identity to the amino acid sequence represented by any of SEQ ID NOs: 2, 4, 6, 8, 10, and 12, and having (i) the activity of converting active jasmonic acid to inactive jasmonic acid or (ii) the activity of enhancing the environmental stress tolerances of plants;
(d) a protein encoded by a polynucleotide consisting of a nucleotide sequence represented by any of SEQ ID NOs: 1, 3, 5, 7, 9, and 11;
(e) a protein encoded by a polynucleotide having 90% or higher identity to the nucleotide sequence represented by any of SEQ ID NOs: 1, 3, 5, 7, 9, and 11, and having (i) the activity of converting active jasmonic acid to inactive jasmonic acid or (ii) the activity of enhancing the environmental stress tolerances of plants; and
(f) a protein encoded by a polynucleotide hybridizing under stringent conditions to a polynucleotide consisting of the nucleotide sequence represented by any of SEQ ID NOs: 1, 3, 5, 7, 9, and 11 or a nucleotide sequence complementary thereto, and having (i) the activity of converting active jasmonic acid to inactive jasmonic acid or (ii) the activity of enhancing the environmental stress tolerances of plants.

10. A plant having a nucleic acid encoding a protein introduced therein or having the increased expression of the nucleic acid, wherein
the protein is selected from the group consisting of:
(a) a protein comprising the amino acid sequence represented by SEQ ID NO: 26;
(b) a protein comprising an amino acid sequence derived from the amino acid sequence represented by SEQ ID NO: 26 by the deletion, substitution, addition, or insertion of one or more amino acids;
(c) a protein comprising an amino acid sequence having 90% or higher identity to the amino acid sequence represented by SEQ ID NO: 26;
(d) a protein encoded by a polynucleotide consisting of the nucleotide sequence represented by SEQ ID NO: 25;
(e) a protein encoded by a polynucleotide consisting of a nucleotide sequence having 90% or higher identity to the nucleotide sequence represented by SEQ ID NO: 25; and
(f) a protein encoded by a polynucleotide hybridizing under stringent conditions to a polynucleotide consisting of the nucleotide sequence represented by SEQ ID NO: 25 or a nucleotide sequence complementary thereto.

11. The plant according to claim 10, wherein the plant has an enhanced environmental stress tolerance.

12. The plant according to claim 1, wherein the plant is a dicotyledon.

13. The plant according to claim 12, wherein the plant is soybean.

14. The plant according to claim 1, wherein the plant is a monocotyledon.

15. The plant according to claim 14, wherein the plant is a plant of the family Poaceae.

16. The plant according to claim 15, wherein the plant is rice, sugar cane or corn.

17.-33. (canceled)
